(19) United States
(12) Patent Application Publication
(10) Pub. No.: US 2020/0261477 A1
(43) Pub. Date: Aug. 20, 2020

(54) Treatment of airway disorders and cough

(71) Applicant: Edward T. Wei, Berkeley, CA (US)

(72) Inventor: Edward T. Wei, Berkeley, CA (US)

(21) Appl. No.: 16/602,539

(22) Filed: Oct. 25, 2019

Related U.S. Application Data
(63) Continuation-in-part of application No. 16/501,056, filed on Feb. 14, 2019.

(51) Int. Cl. A61K 31/662; A61K 9/00; A61P 11/00

(52) U.S. Cl. CPC A61K 31/662; A61P 11/00; A61K 9/007

(57) ABSTRACT

The present discovery pertains generally to the field of therapeutic compounds. More specifically the present discovery pertains to a particular 1-di-alkyl-phosphinoyl-alkane, 1-(Diisopropyl-phosphinoyl)-nonane, referred to herein as “DIPA-1-9”. DIPA-1-9, is able to selectively treat (e.g., suppress) sensory discomfort arising from the airways without side effects. Compared to structurally similar compounds, DIPA-1-9 did not have the problems of excessive cold, stinging, or irritancy, or of adverse taste. To deliver the DIPA-1-9 to the upper airway it is formulated as a solution of DIPA-1-9 in water, a water-based solution, or syrup, at a concentration of 2 to 10 mg/mL and a delivery volume of less than 0.5 mL per unit dose. The drops of DIPA-1-9 are administered into the nasal cavity or onto the base of the tongue, next to the pillars of fauces. The DIPA-1-9 then reaches the nerve endings at the base of the epithelia and transduces signals of coolness and cold. Cooling of the upper airways relieves discomfort and is useful for conditions such as throat irritation, cough, pharyngitis, and lower airway blockage disorders. The elicitation of cooling in the upper airways can be used to control cough, to treat dyspnea, and to enhance mucus clearance in lower airway disorders such as chronic obstructive pulmonary diseases (which includes bronchitis and bronchiectasis), asthma, interstitial lung diseases, cystic fibrosis, lung fibrosis, pneumonia, and other lung disorders. The efficacy of DIPA-1-9 in treating chronic obstructive pulmonary disease is especially attractive because the four primary signs of chronic obstructive pulmonary disease, cough, excessive sputum production, dyspnea, and psychic distress from the lack of control of airway discomfort, are favorably ameliorated by topical application of DIPA-1-9 to the upper airways. This method of treating chronic obstructive pulmonary disease with a cooling agent to the upper airways has not been previously described.

### CROSS-REFERENCE TO RELATED APPLICATIONS

This application is a continuation-in-part of Ser. No. 16/501,056 filed on Feb. 14, 2019.

### BACKGROUND OF THE INVENTION

The US FDA identifies a new medicine as “First-in-Class” when the drug uses a new and unique mechanism of action for treating a medical condition. First-in-Class designation is one indicator of the innovative nature of a drug. For a molecule to succeed as a drug, it is necessary to define the medical condition precisely and to choose the right mechanism of action, the right molecule, the right place (target) for delivery, a delivery system to deliver the right dose at the target site, and to deliver the molecule at the right time. If any of these parameters fail, the new medicine will not work.

### Medications and Target Surfaces of the Airways

The lumen of the airways and the digestive tract is a common conduit for food, liquid, and air, and is part of both the respiratory and digestive systems. This tract is composed of the mouth, pharynx, larynx, airways, and parts of the esophagus. In laymen's terms it includes the organs and tissues of the lips, mouth, tongue, throat, vocal cords, windpipe, lungs, and parts of the esophagus. The traffic that passes through this tract every day is astounding. On an average day, an adult breathes 12,000 L of air, drinks 2 L of fluids, secretes 1 L of saliva, and eats 2 kg of food. These activities are constant, with about 15 breaths and 1 swallowing movement per min during the waking hours. For survival, the traffic flow must be co-ordinated so that food and liquids go down the esophagus and not into the airways, and air gets directed into the airways. The efficiency of this system is visible and self-evident, for example, when a large pizza is consumed with a soft drink. The transit of mass from mouth to stomach is accomplished with a minimum of fuss with the subject breathing at the same time.

The aerodigestive tract lining is susceptible to injury because of exposure to physical, chemical, and biological agents. Refluxed digestive enzymes and acids, infectious agents such viruses, and nasally secreted and airways secreted exudates can all injure the lining, cause inflammation, be a source of purulence, and produce discomfort and pain. A common pathway for the expression of discomfort is cough, as the aerodigestive tract attempts to get rid of the irritant.

A number of menthol-related compounds with physiological cooling effects on epithelia such as the skin and the tongue have been described by Watson et al. [“Compounds with the Menthol Cooling Effect”, J. Soc. Cosmet. Chem. 29: 185-200, 1978]. Some of the compounds are used as additives to toothpaste, cosmetics, and comestibles such as hard candy, but none are used for the medical treatment of the lower airway disorders. Trialkylphosphine oxides having a “physiological cooling action” were described [Rowsell et al. “Phosphine oxides having a physiological cooling effect”, U.S. Pat. No. 4,070,496, 1978]. These compounds were not developed for commercial use. Wei has proposed use of certain water-insoluble cooling agents for the treatment of cough (U.S. Pat. Nos. 8,426,463 and 8,476,317), but these agents are not easily formulated for delivery to nerve endings of the oropharynx. Recent research has focused on drugs that antagonize certain purinergic receptors located on vagal afferents. These new drug candidates must be administered via the bloodstream to reach the 10^{th} nerve endings in the airways.

Current medications for coughing have limited efficacy, as witnessed by individuals who stay awake at night, unable to sleep because of cough, and individuals who cough for prolonged periods, for example, for >3 weeks after a viral infection of the upper airways. The current anti-tussive agents include: honey, dextromethorphan, diphenhydramine, benzocaine, I-menthol and codeine. There is need for a new medication, simply applied, that will immediately control airway discomfort for at least three to four hours to allow the patient to stop coughing and go to sleep. The new agent must have a rapid and robust onset of action, of less than several minutes, to encourage patient adherence. It must be easy to administer and suppress sensory discomfort from the airways without aversive tastes, irritancy, pain, or toxicity. The drug is preferably topically applied and acts locally on the target nerve endings, and must also have a sufficient duration of action to be clinically meaningful.

The airways are divided into upper and lower with the separation of the halves occurring at the glottis (FIG. 1). The dysesthesia arising from the aerodigestive tract differs from that of the skin. Note, for example, the sharp reaction of the laryngeal and tracheal membranes to distilled water; the choking sensations of chili pepper in the throat; the sour, acrid feeling of regurgitated acid in the back of the mouth and throat; the bilious nature of a full meal; the itch and urge to cough; the inability to breathe comfortably; and a throbbing sore throat. The globus of mucus accumulation. These sensations are clearly different from what can be felt from the skin, and each has their own characteristics. The nerve endings that report noxious signals from the aerodigestive tract originate mostly from the trigeminus (5^{th}), glossopharyngeus, (9^{th}) and vagus (10^{th}) nerves, and from some spinal sensory afferents of the esophagus. The targets for drug delivery are to the receptive fields of these nerve endings within the basal layers of the stratified epithelium and respiratory epithelium. The proposed action of the drug of the preferred embodiment is to simulate the sensations of heat abstraction, that is, to cool.

Current medications for lower airway disorders are targeted for delivery to targets in the lower airways. Hence, muscarinic agonists such as tiotropium, long-acting β-adrenergic agonists such as salbutamol, and anti-inflammatory corticosteroids such as fluticasone are targeted at bronchial glands, bronchial smooth muscles, and inflammatory cells within the lower airways. Drugs to enhance mucus clearance, such as mucolytics, and substance to affect mucus secretion or composition, are also targeted for delivery within the lower airways.

### BRIEF SUMMARY OF THE INVENTION.

For successful drug treatment of a medical condition, it is necessary to precisely define the medical condition and to choose the right mechanism of action, molecule, place for delivery, dose, and time of dosing to achieve successful therapy. Here, the medical conditions treated are diseases of the lower airways. Specifically, the sections of the respiratory tract of interest are the obstructed lower airways in the conditions known as chronic obstructive pulmonary blockage disease and asthma.

The novelty of this invention is that the site of drug delivery for treatment is to the upper airways. An agent is applied to the upper airways to treat dysfunctional conditions of the lower airways. Another aspect of novelty is a robust, rapid onset of drug action with a simple method of application, using drops delivered to the back of throat. Another aspect of novelty is that the goal is not just an anti-irritant drug action to suppress cough frequency, but the goal is to allow the subject to control the urge to cough. This is accomplished because of the ease of use, rapid onset, and the robust sensory effect of the active ingredient. Another aspect of novelty is that control of the urge to cough allows the subject to manage and produce an “efficient” cough: that is, a timed cough to efficiently remove excess mucus from the airways. Another aspect of novelty, is that breathing discomfort from the lower airways disorders can be ameliorated by providing a cooling sensation to the upper airways. Finally, it is noted that cough suppression, control of the urge of the cough, an efficient cough, and fresh cool breathing, will help the subject regain psychical confidence in their ability to cope with airway blockage disease.

Broadly, the present discovery provides methods to control a lower airway disorder. Thus, in one aspect of the invention, a therapeutic method for the treatment of a lower airways disorder in a subject in need of such treatment is provided, comprising: topically applying a therapeutically effective amount of 1-[Diisopropylphosphinoyl]-nonane to the upper airways, the therapeutically effective amount of 1-[Diisopropyl-phosphinoyl]-nonane being dissolved in a pharmaceutical vehicle. More preferably, the vehicle is adapted for focused delivery of the 1-[Diisopropyl-phosphinoyl]-nonane to the oropharynx.

Specifically, the sections of the respiratory tract of interest are the obstructed lower airways in the conditions known as chronic obstructive pulmonary blockage disease and asthma. One novelty of this invention is that the site of drug delivery for treatment is to the upper airways to treat dysfunctional conditions of the lower airways. Currently, drug medications for the lower airways are delivered directly to the lower airways.

In treatment of lower airway blockage disorders, the key steps to recognize are the cardinal signs and symptoms of such disorders. These are cough, increased sputum production, loss of lung and gas exchange functions (for example, as measured by spirometry or blood oxygenation levels), dyspnea, and psychic manifestations of breathing disorders such as anxiety, depression, and panic attacks. Coughing and the loss of control of the urge to cough, shortness of breath, and the loss of the ability to breathe comfortably, anxiety and the loss of ability to sleep well, all contribute to the loss of well-being of the subject. By applying a cooling/anti-irritant agent to the upper airways, using the preferred embodiment, we propose the urge to cough can be controlled and the subject can be taught to improve mucus clearance by coughing more efficiently. The control of the breathing process is further enhanced by administering a cooling agent to the nasal cavity to produce a sense of fresh air flow. This will counteract dyspnea. These drug applications to the upper airways can thus relieve symptoms, and therapeutically improve the psychologlical well-being of the subject with lower airway blockage disease, as well as improve lung function.

The right molecule is chosen from a set of agents that selectively and specifically cool (mimic the sensations of heat abstraction) without the adverse effects of bad taste or pain, and have a sufficient duration of therapeutic action. The right place for delivery is the nerve endings of the nasopharyngeal and oropharyngeal epithelium in the upper airways To achieve the right concentration, the molecule is preferably formulated in water or in a water-based solution, or syrup and is topically applied. The volume of liquid is less than 1 mL per dose, and preferably less than 0.5 mL. These are liquid “drops”. The drops used in this manner are a vehicle for focused delivery of the cooling ingredient to the nasal cavity or to the oropharyngeal rim, at the base of the tongue next to the pillars of fauces, so that the active ingredient adheres to the wall of the orpharynx, hypopharynx, and upper third of the esophagus, and is not rapidly transferred into the lower esophagus (see FIG. 3). The drops may contain an artificial sweetener to mask the bitterness of the active ingredient. The sweetener is not used here for its sweet taste to treat cough.

The delivered “drops” act topically on the upper airways and do not enter the lower airways. But the blockage disease occurs in the lower airways.

The placement of the drops in this invention is in the back of the mouth at the base of the tongue (FIG. 3). The taste buds for sweetness are in the front ⅓ of the tongue (anterior), and can be utilized to mask unpleasant tastes. The time of delivery of the active ingredient dissolved in the drops is selected when there is a need to relieve the discomfort. The drops can be administered repeatedly without desensitization. One molecule is chosen from a set of agents that selectively and specifically cool (mimic the sensations of heat abstraction like a spoonful of Häagen-Dazs ice cream) without adverse effects of bad taste or pain. The targeted place for delivery is the nerve endings embedded in the stratified epithelium of the nasal, pharyngeal and esophageal epithelia. To achieve the right dose (which is concentration of the molecule x the volume delivered) the molecule is formulated so that it can be topically applied to and to quickly reach its target. This is achieved with a small volume (≤1 mL) of drops (water or syrup) applied to the base of the tongue, or to the nasal cavity (saline solution). The time of delivery is chosen when there is discomfort and the onset of relief (≤2 min) can be immediate. This timing provides instant relief to the patient and motivates patient adherence or compliance to use of the medication.

The present discovery pertains to a set of molecules called di-alkyl-phosphinoyl-alkane with one particularly preferred entity called 1-(Diisopropyl-phosphinoyl)-nonane, referred to herein as “DIPA-1-9”. Surprisingly and unexpectedly, DIPA-1-9, topically applied to the upper airways is able to treat (e.g., suppress) lower airway disorders selectively and specifically.

By selectivity, it is meant that DIPA-1-9 is first able to act on the TRPM8 receptor but not on TRPV1 or TRPA1 receptors. TRPM8 receptors are associated with the perception of coolness and cold. TRPV1 or TRPA1 receptors are associated with the perception of pain. When compared to structurally similar compounds, selectivity was also found for comparisons of taste measurements on the tongue. DIPA-1-9 had less significant adverse taste than compounds with 6 to 8 carbons in the longest alkyl sidechain (hexyl, heptyl, and octyl). The adverse tastes are described as having “brackish” and metallic qualities.

By specificity, it is meant that DIPA-1-9 (and related analogs) activates the TRPM8 receptor with a range of potencies and full efficacy, as measured by the median effective dose (EC₅₀). The EC₅₀ potency is one aspect of specificity. Another aspect of specificity called “efficacy” is of considerable importance for mechanism of action and for the selection of the right molecule. By efficacy is meant the maximal intensity of the desired pharmacological effect that is attainable. As described herein, DIPA-1-9 is able to evoke cooling sensations on the nasal, pharyngeal and esophageal surfaces that are therapeutically comfortable and beneficial and which are not accompanied by adverse taste, pain or other undesirable sensations. The particular efficacious endpoint that is desired is the coolness that is similar to the sensations of a spoonful of a rich ice cream when swallowed, but longer-lasting.

The “right” dose (concentration x volume of delivery) of the efficacious molecule to activate the receptor is determined by method of drug delivery and physicochemical properties of the candidate molecule to penetrate barriers, and to reach the receptor. After delivery, the residence time of the molecule at the receptor is also a determinant of the “right” concentration. The key structural modifications in the preferred embodiment, DIPA-1-9, is the diisopropyl substitution and the extension of the longest alkyl chain to nine carbons (nonyl). This was learned by experiment. The DIPA-1-9 is 10×more water soluble than some of those of the prior art, and the nonyl substitution prolongs activity. The water solubility allows complete miscibility with the polar carrier vehicle and facilitates delivery.

Another aspect of the present discovery pertains to use of the 1-diisopropylphosphinoylalkanes in the manufacture of a medicament for treatment of diseases, as described herein. The diisopropyl configuration makes the molecule achiral whereas the analogs described in Rowsell and Spring ('496) were ≥95% chiral. A person skilled in the art who examined the prior art would not have routinely noticed the absence of information of the diisopropyl analogs, or be motivated to synthesize and test them. It would have been difficult to predict the dramatic change in water solubility, selectivity, and specificity. Furthermore, it would not have been possible to predict, to infer, or to find that extension of the longest chain to the nonyl group will make significant differences in optimization of selectivity amd efficacy or specificity (the right degree of cold), and of the duration of action.

In the present discovery, DIPA-1-9 “drops” are applied onto the surface of the nasal cavity or onto the oropharynx. The liquid “drops” work by creating a cooling/anti-irritant sensation (via TRPM8 receptors) on the oropharynx surface or in the upper airway. In less than 2 min, the urge to cough is suppressed. A TRPM8 cooling agent applied to the upper airways elevates the threshold for the cough stimuli emanating from the lower airway. The patient can be easily taught by the health practioner to control the urge to cough with DIPA-1-9 drops. The drops reduce cough frequency and allay anxiety because the patient now know how to control the cough and reduce the discomfort in the throat.

Once the urge to cough is controlled by the patient, the next step for the patient with productive or wet cough is to learn how to utilize the DIPA-1-9 drops to effectively clear mucus/phelgm from the airways. Many coughs are hacking, painful, and inefficient: i.e. they do not clear the airways of secretions or mucus. By controlling the urge to cough, however, the subject learns how to let the cough and sputum accumulate, and then in one efficient cough, expectorate the phlegm.

If the patient with chronic obstructive lung disease has dyspnea, further delivery of DIPA-1-9 drops to the nasal cavity will counteract dyspnea. Controlling cough, mucus clearance, and difficulties in breathing will make the patient feel better. Anxiety, insomnia and depression are diminished, and there is therapeutic benefit.

Another aspect of the present discovery pertains to use of a small volume of drops (≤1 mL) for the delivery of the active ingredient, for example DIPA-1-9. to the oropharyngeal surface. The rapid transit time of a bolus (35 cm/sec) pass the oropharynx hinders any contact time with the nerve endings of the pharynx. The pharyngeal transit time is <1 sec, and averages ˜0.5 sec. Using a medication dissolved in saliva requires constant secretion of saliva and swallowing to coat the pharynx, and is not convenient. The drops formulation, e.g. in just water, a water-based solution or a syrup, achieves excellent results. The drops provide a homogeneous distribution system for DIPA-1-9 at its precise desired site of action and has an immediate onset of effect. As will be appreciated by one of skill in the art, features and preferred embodiments of one aspect of the discovery will also pertain to other aspects of the discovery.

The inventive step is to use upper airway targets to control a lower airway disorder. To my knowledge, this type of scientific rationale and the mechanistic description of implementation for drug action are not in the prior art.

### BRIEF DESCRIPTION OF THE SEVERAL VIEW OF OF THE DRAWINGS

FIG. 1. shows a diagram of the human airways with separation of the upper airways and lower airways at the level of the glottis (sold black line). The inventive step is to topically delivery a TRPM8 agonist to the upper airway in order to ameliorate the signs or symptoms of a lower airway blockage disorder.

FIG. 2. is a drawing of the innervation of the human pharynx, demonstrated by the Sihler's stain. The drawing is adapted from Mu and Sanders, “Sensory nerve supply of the human oro- and laryngopharynx: a preliminary study.” Anatomical Record 258:480-420, 2000. The nerve endings of the upper oropharynx are primarily from the 9^{th} nerve (glossopharyngeus), and the nerve endings for the laryngopharynx from the 10^{th} nerve (vagus). The lateral and posterior walls of the oropharynx are innervated by both the 9^{th} and 10^{th} nerves. Epi=epiglottis, medium black areas=tonsils, and the small black areas are lymph granules. These sensory nerve endings transduce the signals from the pharynx to the brain and coordinate sensory perception and muscular response.

FIG. 3. is a drawing of the human oral cavity and show the target area for placement of the DIPA-1-9 drops (black outlined circle), at the base of the tongue. The sensory nerve endings for the detection of coolness are abundant in the upper oropharynx and also at the bases of the anterior arches of the pharynx, called the pillars of fauces. The delivery of the DIPA-1-9 drops to the target site transduces signals of coolness from the pharynx to the brain and is also anti-irritant.

FIG. 4. is a graph of fluorescence response (Δ ratio 340/380) in TRPM8 transfected cells as a function of the logarithm of the concentration of the test compound, expressed in μM, for DIPA-1-7 (black circle), 3,4-7 (open squares), or 3,4-6 (open triangles). The assays were conducted by Andersson et al. of King's College, London, UK, using his methods described in “Modulation of the cold-activated channel TRPM8 by lysophospholipids and polyunsaturated fatty acids. Journal Neuroscience 27 (12): 3347-3355, 2007.

FIG. 5. shows a comparison of the sensory effects of DIPA-1-7, DIPA-1-8, and DIPA-1-9 administered to the base of the tongues of 4 volunteers using a 2 mL vial for delivery. The DIPA compounds was 5 mg/mL in cherry-flavored syrup in a volume of 0.8 mL per dose. The sensory effect were recorded every 5 min for 1 hr.

FIG. 6. shows DIPA-1-9 inhibits cough frequency in a mouse model of respiratory tract viral infection. Mice (n=4 to 6 per group) cough more frequently (black bars) after inoculation with respiratory synctial virus (RSV). Codeine administered 1 mg perioral (p.o.) per mouse, or DIPA-1-9 0.5 mg in 25 μL intranasally (i.n.) per mouse, significantly inhibited cough frequency (*P≤0.01 and ≤0.05 for the three time periods of testing, Dunnett's test for multiple comparison). These results in mice show that DIPA-1-9 has antinociceptive activity in the upper airways.

### DETAILED DESCRIPTION OF THE INVENTION

Molecular Mechanism of Action. The structure and function of the cooling and of anti-irritant mechanisms of drug action are first described. TRPM8, the molecular target of drug action is an integral membrane protein that responds to heat abstraction (<25° C.) by opening a cation channel. If the channel is on a neuronal membrane, the opening of the channel results in depolarization. The actions potentials generated enter the central nervous system and is interpreted by the brain as coolness, an anti-irritant action if nociception is present, and a greater awareness of the topographical origin of the stimulus. The structural biology of TRPM8 has recently been elucidated by cryo-electron microscopy (Ying et al. Science 359: 237-241, 2018) and the sites for chemical agonism and antagonism were identified.

The mechanisms of signal transduction for TRPM8 are quite intriguing. The entry of cations past a cell membrane triggers an action potential. This TRPM8 action can be demonstrated in biplanar lipid layers containing TRPM8, without intracellular metabolic machinery such as mitochondria or DNA. Action potentials are increased in number as temperature drops from 20° C. to 15° C. These action potentials are transmitted to the brain and can be recorded as neuronal spikes in brain nuclei. The frequency, duration, and topographical origin of the signals determine the brain's interpretation of the transmitted information. The brain perceives the TRPM8 signals as a) a drop in the temperature differential, b) in the presence of an irritant, coolness diminishes irritation, c) coolness increases awareness of inputs originating from the location of the signals, and d) coolness signals plus proprioceptive signals (via mechanoreceptors such as Piezo2) are perceived as wetness.

Normally, physiological activities such as breathing, mastication of food, perception of thirst, swallowing a bolus of liquid or solid, mucus clearance, and cough use these TRPM8 signals to maintain function of the airways and digestive tracts.

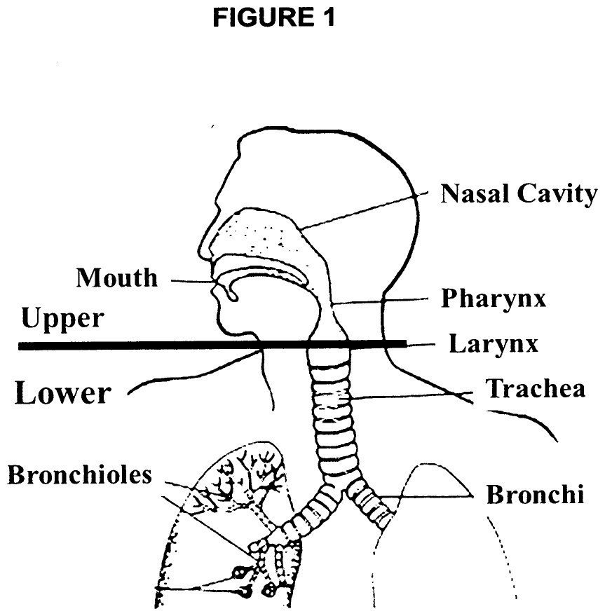

Anatomically (FIG. 1), the airways are divided into upper and lower portions at the level of the glottis (solid black line). TRPM8 receptors are primarily present in the upper airways and have physiological functions in breathing, mastication, swallowing, drinking, mucus clearance and cough. The lower airways do not have or need a TRPM8 sensory apparatus to detect coolness. Heating of inspired air by blood flow is very efficient. When warm (+26.7° C.) or frigid (−18.6° C.) air are inspired by humans, the air temperature is already at 25 to 30° C. when it reaches the glottis (McFadden et al, J Appl Physiol 58:564-70.1985). This warmed-up air at the level of the glottis is above the activation temperature for TRPM8.

The TRPM8 serve as temperature detectors for coolness, and activation by lower temperatures result in an anti-irritant effect. Coolness also increases awareness of mechano-sensations, and can convey a sense of “wetness”. The sensations of wetness helps quench thirst and may aid in the awareness of mucus clearance from the airways. The threshold for TRPM8 activation is a temperature <25° C. Inspired air is normally heated to >20° C. by the time the air reaches the glottis. So the absence of TRPM8 sensors in the lower airways is understandable.

In lower airway disorders, there is inflammation of the airway walls, increased secretions, and hypersecretion of mucus. The secretions are wafted up the bronchi, into the trachea, then into the glottis as phlegm. The clinical results of the lower airway disorders are cough, increased sputum production, and dyspnea (in more severe cases because of the blockage), and the difficulties in breathing sensations may cause anxiety and panic attacks. Surprisingly and unexpectedly, activation of TRPM8 receptors with an agonist such as DIPA-1-9 in the upper airways can ameliorate the signs and symptoms of lower airway blockage disorders. The cooling/anti-irritant actions from a TRPM8 agonist enables suppression and control of cough frequency. Control of cough and throat discomfort enables the deliberate clearance of accumulated mucus by the patient. Administration of DIPA-1-9 will give a sense of refreshed breathing and reduced dyspnea. Together, these pharmacological actions of the TRPM8 agonist give the patient a better sense of controlled, comfortable breathing, enable better sleep, and improve the psyche of the patient so there is less anxiety and depression.

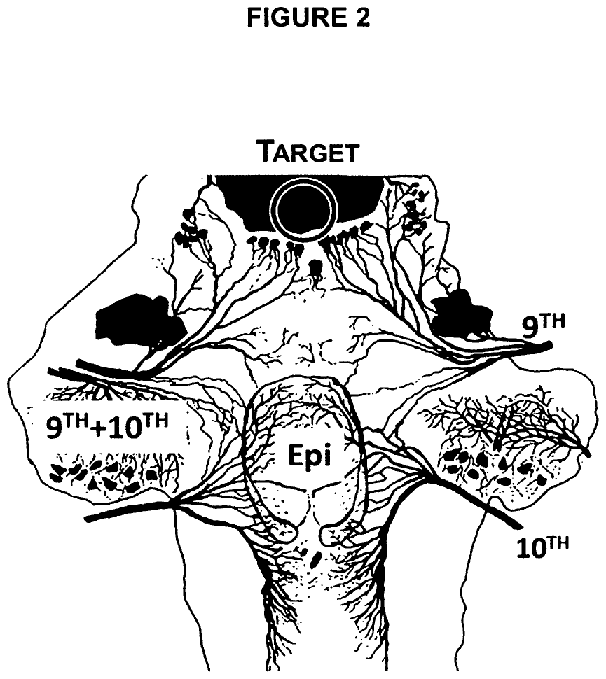

The receptive field of the TRPM8 nerve endings are located on 9^{th} and 10^{th} nerves, especially on the upper margins of the oropharynx and on the lateral walls of the oropharynx (FIG. 2). The TRPM8 receptors are not present around the epiglottis (Epi), innervated by the 10^{th} nerve. The scarcity of TRPM8 nerve endings in the lower airways is clearly demonstrated in this study of Hondoh et al. (Brain Res. 1319:60-9, 2010). The neuronal cell bodies of the 10^{th} nerve are located in the nodose ganglion (NG). The neuronal cell bodies of the 9^{th} nerve are in the jugular (JG) and petrosal ganglia (PG). Using an anti-sense method, Hondoh et al. showed that the TRPM8 cell bodies are located in JG and PG, but not NG. By contrast, TRPA1 containing neurons are located in all three ganglia. By inference, the nerve endings of the 10^{th} nerve contain little TRPM8 receptors, and the nerve endings surrounding the glottis do not convey messages of coolness.

Menthol lozenges have been around since the 1930s, and are sold by Halls (Mondelez Global, Canada) at Walmart stores for about $2 per bag of 30. The lozenges are called “hard candy” and each weigh about 3 g. When the lozenge is in the mouth, it is mechanically impossible to cough. The saliva dissolves the lozenge and there is a cooling effect in the oral cavity and throat because these lozenges contain menthol at levels of 2.5 to 16 mg. The limitations of the lozenge are the harsh taste of menthol, and the need to hold the lozenge in the mouth till it completely dissolves (˜30 min). For the higher doses of menthol, there is cold discomfort in the chest behind the sternum. This is an unpleasant sense of coldness behind the sternum is frightening to some patients because chills remind people of death. Most likely, it is the menthol dissolved in the saliva that is acting on the esophageal lining. The fast pharyngeal transit time (PTT) of ≤1 sec prevents retention of the mentholated-saliva on the upper airway surface. So as soon as the lozenge has completely dissolved, any salutary effects of menthol on the throat also dissipate.

There is no evidence that menthol lozenges are used for therapy by patients with lower airway disease.

By contrast, the DIPA-1-9 delivered in a few drops, at a volume of ≤0.5 mL, gives an intense cooling sensation that is powerful and has a duration of action ≥2 hr in the suppression of cough. The drops are superior to the lozenge as a method of delivery.

In lower airway disease, a key feature of pathophysiology is mucus hypersecretion and accumulation. This occurs, for example, for COPD, certain types of asthma, pulmonary fibrosis, cystic fibrosis, lung cancer, pneumonia of diverse origins, and viral and bacterial infections. The mucus physically obstructs smooth airflow, is a source for growth of micro-organisms, and interferes with gaseous exchange (Rogers, 2006). In the airways, the secretions are called phlegm, and when it is expectorated it is called sputum. A primary objective of therapeutic treatment is to efficiently remove the mucus and phlegm by efficient coughing.

In lower airway blockage diseases efforts to clear phlegm can be laborious and painful, because the airways surface is inflamed. The only two ways to clear phlegm are by coughing or changing mucus rheology. The muscular effort to clear by cough wears down the patient's energy. With DIPA-1-9 drops, there is increased awareness of the phlegm in the throat and patients can be taught to sense mucus accumulation at timed intervals, e.g. 5, 10, 15 min. The drops allow patient to “control” sense of throat discomfort, and to manage ineffective coughing “fits”. The subject is taught to accumulate the mucus in the airways, brace themselves and give a coordinated cough that can use the force of a cough (air flows of up to 280 m/sec) to expectorate. This technique can be readily taught by a health practioner to the patient.

Shortness of breath and a sense of suffocation (dyspnea) are pathognomonic signs of advanced lower airway disease. Patients report that breathing cold air sometimes relieves this discomfort. Instillation of DIPA-1-9 drops into the nasal cavity will give a sense of fresh airflow. One subject who tried it described it as “breathing in clean mountain air on a clear crisp morning”. Breathing the air in the hills near the Golden Gate Bridge, on a foggy day, will also provide this experience of “clean, cool breathing.” This event can be reproduced with DIPA-1-9 drops instilled into the nasal cavity at a concentration of 1.0 to 1.5 mg/mL of saline. The nasal TRPM8 receptor target sits on the mucosal surface and is located near the nasal valve.

Measurement of Therapeutic Efficacy. The utility of DIPA-1-9 drops can be quantified in standard tests of airway function. For example, cough counts can be measured using the Leicester Cough Monitor, which is a personal microphone attached to a recorder, with appropriate software for counting coughs. Expectoration of mucus can be quantified by collecting sputum in a cup and measuring the mg mucus over time. Improvement of lung function can be quantified by spirometry. The best indicator of efficacy is, however, an improvement in the patient's numerical rating scale (NRS) of the benefits of medication on a scale of 1 to 10 (with 10 being the worst condition). Efficacy is shown when the NRS averages goes down significantly when treatment is compared to a placebo control under randomized, double-masked conditions.

Spirometry (meaning the measuring of breath) is the most common of the pulmonary function tests (PFTs). It measures lung function, specifically the amount (volume) and/or speed (flow) of air that can be inhaled and exhaled. Spirometry is helpful in assessing breathing patterns that identify conditions such as asthma, pulmonary fibrosis, cystic fibrosis, and COPD. The most common parameters measured in spirometry are Vital capacity (VC), Forced vital capacity (FVC), Forced expiratory volume (FEV) at timed intervals of 0.5, 1.0 (FEV1), 2.0, and 3.0 seconds, forced expiratory flow 25-75% (FEF 25-75) and maximal voluntary ventilation (MVVV), also known as Maximum breathing capacity.

Shortness of breath or dyspnea can be measured by standardized methods, including questionnaires and spirometry, which are described in the 2019 GOLD (Global Initiative for Lung Disease Report), pg. 30-35. The full report is 155 pages, is incorporated herein by reference, and can be downloaded.

In summary, I have proposed a new strategy for treating lower airway blockage disorders, using topical delivery of a set of cooling agents to the upper airways. For the lower airway disorder of COPD, the signs and symptoms to be alleviated are cough, excess sputum production, and dyspnea. The relief of dyspnea by cooling is to enable the patient to have psychic control over the sensations of the urge to cough, shortness of breath, dyspnea, and sense of suffocation. If control is established, anxiety, depression, and panic will subside and the incidence of exacerbations and hospitalization should decrease. Exacerbations (a worsening of respiratory function) are important economic burdens and the average costs per exacerbation is about $10,000 per incident.

The present discovery pertains to the selection of 1-dialkylphosphorylalkanes for the treatment of lower airway disorders. In particular an active ingredient called 1-Diisopropyl-phosphinoyl-nonane and referred to herein as “DIPA-1-9” was especially effective. DIPA-1-9, is rapidly able to treat (e.g., selectively suppress) sensory discomfort from the lower airways by topical application to the surfaces of the upper airways, without problems of side effects. The urge to cough is suppressed, clearance of mucus is facilitated, dyspnea is alleviated, and the subject feels better. The onset of effect is immediate (≤2 min) and surprising. There are no other products on the market that match this rapid action. Consequently, DIPA-1-9 is useful, for example, in the treatment of lower airway blockage disorders such as, cough, excess sputum production, dyspnea, and psychic dysfunction of airway disorders. The present discovery pertains to pharmaceutical compositions comprising DIPA-1-9, and the use of DIPA-1-9 compositions, for example, in therapy.

As described herein, the Inventor has defined the logic of choosing the receptor target, the mechanisms of action, target surfaces, screening methods, bioassays, and animal models for treating the lower airway disorders and identified methods for drug delivery to the upper airways that will enable control of the lower airway symptoms and signs.

The preferred embodiment, referred to herein as DIPA-1-9, had an ideal combination of properties for the medical treatment of the surface of the upper airways. As described in the studies below:
The diisopropyl substitution on the molecule makes the DIPA entities more water soluble and enables delivery to the surfaces of upper airways as “drops”: that is, a liquid formulation in water, in isotonic saline, or in syrup.
The formulation of the drops in a small volume of liquid (˜0.1 to 0.5 mL per dose) is easy to use and allows precise delivery and dosage of DIPA-1-9 to the target nerve endings of the upper airway surface (nasal cavity and oropharynx).
DIPA-1-9 evokes a rapid pleasant strong cooling sensation on the throat, but without unpleasant taste or pain.
A precise definition of the cooling action of DIPA-1-9 drops, is analogy to the swallowing of a spoonful of a rich ice cream, such as Häagen-Dazs ice cream. This sensation allowed it to be differentiated from other analogs which produced cold discomfort.
DIPA-1-9's cooling sensations are sufficiently prolonged [≥15 min and up to >2 hr] to be of therapeutic benefit in multiple indications. Application to the throat also leaves a residual antinociceptive effect that lasts ≥2 hr.
The unusual properties of DIPA-1-9 could not have been predicted based on its TRPM8 receptor activation potency, but had to be discovered by experiment. There is no simple correlation between the EC₅₀ [measurement of TRPM8 potency] and efficacy for activity on the airway uses.
DIPA-1-9 inhibited cough frequency in a respiratory syncytial virus induced model of cough in the mouse.
When tested in volunteers with cough, sputum production, and uncomfortable breathing, DIPA-1-9 drops had a rapid onset of action of ≤2 min and alleviated discomfort without adverse effects.
No current medications for treatment of lower airway blockage disorders has such properties of rapid onset and efficacy.
No current medications rationalizes the treatment of lower airway blockage disorders with delivery of a cooling agent to the upper airways.

These results, in multiple test systems, show that the preferred embodiment DIPA-1-9 exhibits unusual selective and specific drug actions. Consequently, DIPA-1-9 is useful, for example, in the treatment of lower airway blockage disorders (e.g., diseases) such as COPD, asthma, pulmonary fibrosis, tuberculosis and lung cancer.

### Abbreviations and Terminology

Upper and Lower Airways. The upper airway is the airway from the nares to the glottis (lips of the larynx). The lower airway is the portion of the respiratory tree that extends from below the glottis to and including the terminal bronchioles. The glottic inlet clearly define the border between the upper and lower airways. Currently, the upper airway is not a drug treatment target for the management of symptoms and signs originating from the lower airways. Here, the target for topical delivery of the preferred embodiment, DIPA-1-9 in drops, is onto the surface of the nasal cavity and on the rostral edge of the oropharynx. Thus, delivery is to the upper airways.

DIPA compounds. DIPA is the abbreviation for 1-[Diisopropyl-phosphinoyl]-alkane. The third alkyl group in the molecule may be described by a number: hence, 4, 5, 6, 7, 8, 9, and 10 correspond to the butyl, pentyl, hexyl, heptyl, octyl, nonyl, and decanyl side chain, respectively. The longest alkane sidechain is linear or “normal [n]” in configuration, with the phosphinoyl group attached to the primary, or “1-” position, of the carbon chain in the third sidechain. These compounds are also known as trialkylphosphine oxides or as 1-dialkylphosphorylalkanes.

TRP channels. The transient receptor potential (TRP) is a family of six cation channels that are integral membrane proteins. These proteins detect thermal, nociceptive and painful signals. Many of these receptors are located on the nerve membranes of sensory neurons and respond to chemical irritants and changes in local temperature by activating nerve action potentials which are inputs to be perceived and acted upon by the brain. The TRP receptors are the transducers of sensory information, and it is this transduction and effector system that regulates and protects the organism from external irritants or temperature extremes.

TRPM8. TRPM8 is a member of the TRP channel family. TRPM8 is an integral membrane protein that responds to heat abstraction (<25° C.) by opening a cation channel. If TRPM8 is on a neuronal membrane, the open channel will generate action potentials that are interpreted by the brain as coolness, an anti-irritant action if nociception is present, and a greater awareness of the topographical origin of the stimulus. The “greater topographical awareness” is especially important for breathing, for swallowing, and for detection of foreign objects or accumulations such as mucus/phlegm. The structural biology of TRPM8 has recently been elucidated by cryo-electron microscopy (Ying et al. Science 359: 237-241, 2018) and sites for chemical agonism and antagonism were identified.

Receptive field. Receptive field of a sensory neuron is the region in space in which a stimulus will modify the firing of the neuron. The receptive field is spatially determined by the distribution of the nerve endings of the neuron. For the epithelium, the nerve endings are interdigitated with the cell layers at the basal layer of the epithelium. A receptive field, even though smaller than a mm², when activated by the appropriate stimulus, e.g. nociceptive or pruritic, can totally dominate the attention of the brain and mind. Witness what happens when a sharp pin or sting comes into contact with skin or when a dog is pre-occupied with a flea bite. To perceive TRPM8 effects, hold an ice cube in the palm of the hand, or drink a glass of cold water at night, when it is quiet and dark. You can feel the activation of the coolness.

Cold Discomfort. One aspect of the discovery here is that many of the compounds tested evoke coolness and cold sensations of different intensities. One level of intense cold is painful to the throat. The sensations are akin to rapid drinking of cold water equilibrated with ice chips. The intense cold is accentuated if the drink is acidified, for example, with lemonade. The sensations of penetrating and intense cold on the surface of the oropharynx are uncomfortable, and aversive. The terms “icy cold” is used to describe this adverse event in the throat. To experience icy cold: Take a glass of water equilibrated, (after stirring) with ice chips—a temperature of about 4° C. Start sipping the water at the rate of about 1 sip per second. The first 5 sips are pleasant, but by 5 to 10 sips, the throat feels a dull cold, and after about 10 to 15 sips, the icy cold in the throat becomes unpleasant, and the sensations of icy cold can be felt in the chest, half-way down to the stomach. These unpleasant sensations constitute “cold discomfort”.

The feeling of cold behind the sternum and in the upper thorax can also be experienced with cooling agents, especially with large doses of I-menthol (>16 mg per candy). These sensations may be accompanied by chills. Most likely, the compound, dissolved in saliva, rapidly distributes and activate cold receptors in the eosophageal lining. The number of cell layers on the esophageal epithelium is less than that of the pharynx. These sensations of cold, if not expected by the test subject, can alarm and be viewed as unpleasant. The substernal chills, normally considered unpleasant, may have some utility in counteracting the discomforts of chest pain.

The two types of “cold discomfort” described here, icy cold and substernal cold, limit selection of the active ingredient for localized action on the oropharynx/upper esophagus. The ideal agent must have a circumscribed site of action, and the intensity of the sensation should not cause “icy cold” in the throat or coldness in the chest.

When ice cream is placed in the mouth, there are pleasant cooling and sweet sensations on the tongue and on the walls of the mouth. When the ice cream is swallowed there is a brief robust refreshing sensation on the back of the mouth. This sensation in the upper throat can be replicated by repetitive swallowing or sipping of ice cream, or the equivalent sipping of a “milk shake” or “smoothie”. An ideal sensation is replicated by swallowing a rich ice cream, such as Häagen-Dazs ice cream which, because of its high cream and low air content, abstracts heat at an optimal rate. This Häagen-Dazs type of ice cream sensation is optimal for treating airway disorders and relieving pharyngeal/esophageal discomfort. This sensation is called an “ideal cool” for reducing aerodigestive tract discomfort.

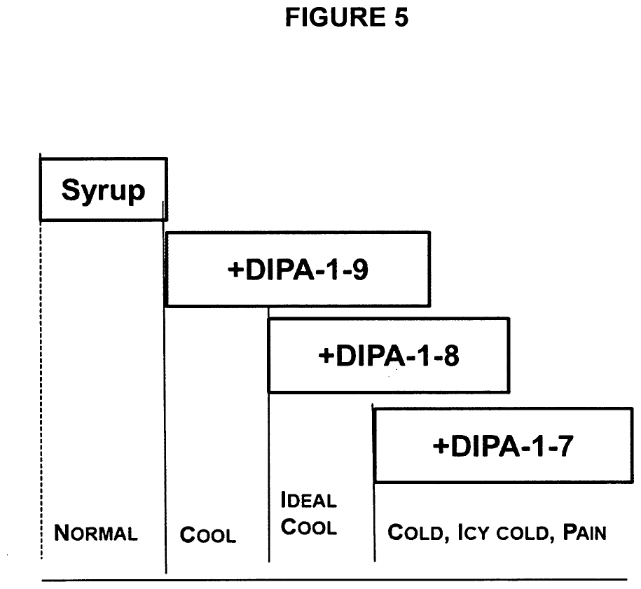

Why are the sensations of sipping ice cream different from that of ice cold water? In both situations, the temperature of the contents in the throat is about the same, yet it is seldom possible to get unpleasantly cold in the throat with ice cream! One explanation is that the thermal conductivity of the oils and fats that make up ice cream is different from water. For example, the thermal conductivity value of olive oil is 0.17 W/m·K and that of water is 0.58 W/m·K. Ice water, with higher thermal conductivity (and higher thermal mass), abstracts more heat than ice cream. The rate of heat abstraction from the surface of the throat is then the determinant of the sensory perception and when it is too rapid or continuous, there is cold discomfort. On the other hand, a smooth heat abstraction rate produces a refreshing sensation. Experimentally, an ice cream with a high cream content, such as Häagen-Dazs vanilla, works best for eliciting ideal cool. The pharmacological goal is then to identify a chemical sensory agent (i.e., a compound that does not abstract heat) that produces an ideal cool and not cold discomfort. Surprisingly and unexpectedly, DIPA-1-9 at the optimized concentrations of 5 to 10 mg/mL elicits an ideal cool in the oropharynx and esophagus but without cold discomfort (FIG. 5).

Drop. The drop is an approximated unit of measure of volume: for example, as the amount dispensed as one drop from a reservoir. An abbreviation for a drop is gt or gtt which come from the Latin noun gutta (“drop”). The volume of a drop is not well defined: it depends on the device and technique used to produce the drop, and on the viscosity, density, and the surface tension of the liquid. Here, the liquid is either water-based (>95% water) or syrup-based. The drop used here is about 0.05 mL per drop or a minim. The unit volumes for delivery to the nasal cavity and the oral cavity are ˜0.20 to ˜0.50 mL per dose, respectively.

### DAPA and DIPA Compounds

The discovery relates to a particular compound within the series of compounds known as phosphine oxides (which have the following general formula), and more particularly, an example of the group known as di-alkyl-phosphinoyl-alkanes (herein referred to as “DAPA compounds”) (wherein each of R₁, R₂, and R₃ is an alkyl group).

(O═)P R₁ R₂ R₃

And more specifically, to the preferred 1-diisopropyl-phosphinoyl-alkane (DIPA) known as 1-Diisopropyl-phosphinoyl-nonane, referred to herein as “DIPA-1-9”.

TABLE 1
Chemical structure of DIPA-1-9

| | Chemical | Formula/ | |
|---|---|---|---|
| Code | Name | Weight | Chemical Structure |
| DIPA-1-9 | 1-Diispropyl- phosphinoyl- nonane | C₁₅H₃₃OP 260.40 | 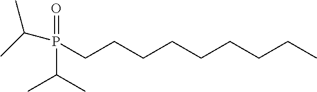 [SMILES: CCCCCCCCCP(=O)(C(C)C)C(C)C] |

DIPA-1-9 is a colorless liquid at room temperature, with a density of ˜0.92 g/cm³ and a boiling point of 112-120° C. Note that DIPA-1-9 is achiral and does not have enantiomers.

By comparison to related DAPA compounds, the Inventor has identified DIPA-1-9 as an exceptional agent for the treatment of sensory discomfort arising from the epithelia, including mucous membranes, of the upper aerodigestive tract, for example, the oropharyngeal (including, e.g., the oropharynx and hypopharynx) and upper esophageal surfaces. The applicant has reported on the efficacy of DIPA-1-9 for the mucous membranes of the nasal cavity and for the transitional epithelium of the ocular surface (U.S. Pat. Nos. 9,642,868 and 9,895,382). This is the first detailed report of the activities of DIPA-1-9 on the aerodigestive tract.

As described herein, DIPA-1-9 is selective and specific and ideal for evoking localized cooling in the oropharynx without discomfort. This refreshing sensation of cool/cold is the desired sensory quality for relieving oropharyngeal/esophageal discomfort. By topical administration the DIPA-1-9 sensation is localized. In an animal model, DIPA-1-9 inhibited virus-induced coughing. DIPA-1-9 is not an irritant in the oral cavity of human volunteers or when it was put into the throat where it exerted the desired antinociceptive effect. The receptive element on neuronal membranes for DIPA-1-9 was further identified as TRPM8, an ion channel receptor. Unlike related analogs, DIPA-1-9 did not produce stinging, or “icy cold” pain, even when the dose was increased to 12 mg per unit.

By choosing drops (water, a water-based solution, or syrup) as a delivery vehicle and a delivery volume of <0.5 mL, the activity of DIPA-1-9 was confined to the throat and upper esophagus, and there was no systemic cooling. The drops was used as a vehicle because, as a liquid, it allowed homogeneous delivery to the pharyngeal surface. Individuals with throat discomfort preferred DIPA-1-9 because of the rapid onset and the pleasant cool sensation. The “icy cold” seen with other DAPA compounds (DIPA-1-6, DIPA-1-7, DAPA-2-6, and DAPA-2-7) was considered not to be optimal, even though these compounds had an equivalent fast onset or were long-acting. The activity of other DAPA compounds (DIPA-1-6, DIPA-1-7, DAPA-2-6, and DAPA-2-7) spreads into the chest, most likely because of activation of sensory elements in the oesophageal lining. This central sternum cooling is perceived by the subject as unpleasant. The duration of action of DIPA-1-9 was sufficient to be therapeutically useful.

### Chemical Synthesis

DAPA compounds were prepared by the following general method: 100 mL (23.7 g, ˜200 mmol) of sec-butylmagnesium chloride or bromide (isopropylmagnesium chloride or bromide) (obtained from Acros, as a 25% solution in tetrahydrofuran (THF)) was placed under nitrogen in a 500 mL flask (with a stir bar). Diethylphosphite solution in THF (from Aldrich, D99234; 8.25 g, 60.6 mmol in 50 mL) was added drop-wise. After approximately 30 min, the reaction mixture warmed up to boiling. The reaction mixture was stirred for an extra 30 min, followed by a drop-wise addition of the appropriate n-alkyl iodide solution in THF (from TCl; 60 mmol in 20 mL). In the case of DIPA-1-9, the n-alkyl halide was 1-iodononane. The reactive mixture was then stirred overnight at room temperature. The reaction mixture was diluted with water, transferred to a separatory funnel, acidified with acetic acid (˜10 mL), and extracted twice with ether. The ether layer was washed with water and evaporated (RotaVap Buchi, bath temperature 40° C.). The light brown oil was distilled under high vacuum. The final products, verified by mass spectrometry, were clear liquids that were colorless or slightly pale yellow.

The compounds prepared by these methods are shown in Table 2.

TABLE 2
Chemicals prepared and tested.

| Code | Chemical Name | Chemical Structure |
|---|---|---|
| DIPA-1-5 | 1-Di(isopropyl)- phosphinoyl- pentane | 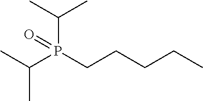 |
| DIPA-1-6 | 1-Di(isopropyl)- phosphinoyl- hexane | 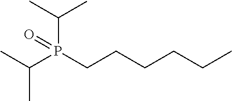 |
| DIPA-1-7 | 1-Di(isopropyl)- phosphinoyl- heptane | 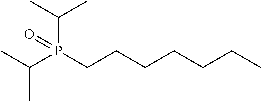 |
| DIPA-1-8 | 1-Di(isopropyl)- phosphinoyl- octane | 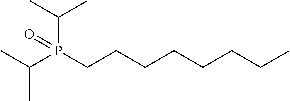 [SMILES: CCCCCCCCP(=O)(C(C)C)C(C)C] |
| DIPA-1-9 | 1-Di(isopropyl)- phosphinoyl- nonane | 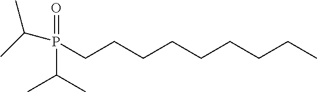 [SMILES: CCCCCCCCCP(=O)(C(C)C)C(C)C] |
| DAPA-2-4 | 1-Di(sec-butyl)- phosphinoyl- butane | 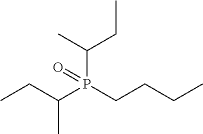 |
| DAPA-2-6 | 1-Di(sec-butyl)- phosphinoyl- hexane | 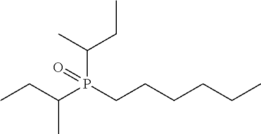 [SMILES: CCCCCCP(=O)(C(C)CC)C(C)CC] |
| DAPA-2-7 | 1-Di(sec-butyl)- phosphinoyl- heptane | 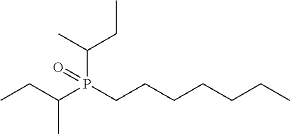 [SMILES: CCCCCCCP(=O)(C(C)CC)C(C)CC] |
| DAPA-2-8 | 1-Di(sec-butyl)- phosphinoyl- octane | 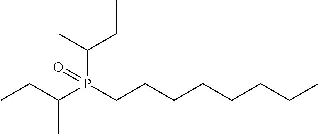 [SMILES: CCCCCCCCP(=O)(C(C)CC)C(C)CC] |
| 3,4-6 | 1-(Isopropyl-sec- butyl)- phosphinoyl- hexane | 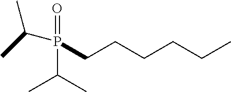 |
| 3,4-7 | 1-(Isopropyl-sec- butyl)- phosphinoyl- heptane | 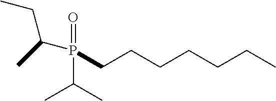 [SMILES: CCCCCCC[P@@](=O)(C(C)C)[C@@H](C)CC] |
| DAPA-3-1 | 1-di(iso-butyl) phosphinoyl- pentane | 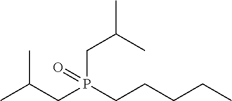 |
| DAPA-3-2 | 1-Di(sec-butyl) phosphinoyl- 3-methyl-butane | 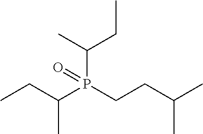 |

Compositions The 3,4-X series are “mixed” isopropyl-sec-butyl compounds (see below). These were synthesized by Dr. Jae Kyun Lim of Dong Wha Pharmaceuticals, S. Korea, using the method described below.

Briefly, as illustrated in the following scheme, triethyl phosphite (A) was reacted with sec-butyl magnesium bromide (B) and then hydrolysed with dilute hydrochloric acid to give the mono-alkyl compound (C). The product (C) was then reacted isopropyl magnesium bromide (D) to give the di-alkyl compound (E), which was then reacted with a suitable alkyl iodide (F) to give the target trialkyl phosphine (G).

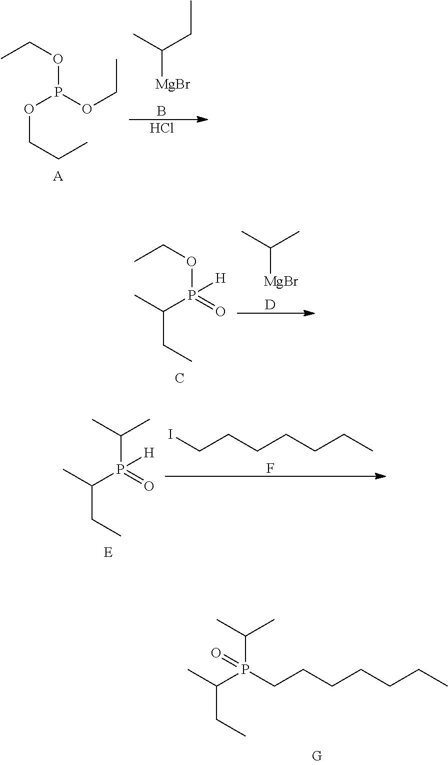

The DIPA compounds are colorless liquids with a density less than water. These structures differ from those described by Rowsell and Spring U.S. Pat. No. 4,070,496 because '496 structures have their “head” (phosphine oxide group) covered by larger, more lipophilic groups. The applicant noted that '496 did not include the di-isopropyl analogs. The applicant synthesized these analogs (which are achiral, by contrast to the structures of '496 which are >95% chiral). The applicant found that, by minimizing the two alkyl side chains to di-isopropyl, the “head” of the prototypical molecule now is more polar (hydrophilic) and more miscible in the polar environment of water. This increased water-solublility is striking (Table 3). The water solubility of the DIPA if at least 10×greater than the di-sec-butyl or the mixed isopropyl-sec-butyl analogs. The DIPA analogs are now mobile in the extracellular fluids and permeate between cells to access nerve endings in the stratum basale.

TABLE 3
Water solubility (mg/ml) of 1-dialkylphosphorylalkanes (R₁R₂R₃P═O).

| | No. Carbons | | | | | | | |
|---|---|---|---|---|---|---|---|---|
| | 13 | | 14 | | 15 | | 16 | |
| R₁, R₂ | R₃ | | R₃ | | R₃ | | R₃ | |
| di-sec-butyl- | pentane | 22 | hexane | 8 | heptane | <3 | octane | <3 |
| isopropyl-sec- | hexane | 25 | heptane | 20 | octane | <3 | nonane | <3 |
| butyl- | | | | | | | | |
| di-isopropyl- | heptane | >300 | octane | >300 | nonane | >300 | decane | <3 |

The discovery also relates to a composition (e.g., a pharmaceutical composition) comprising DIPA-1-9, and a pharmaceutically acceptable carrier, diluent, or excipient. The discovery also relates to a method of preparing a composition (e.g., a pharmaceutical composition) comprising mixing DIPA-1-9, and a pharmaceutically acceptable carrier, diluent, or excipient.

In one embodiment, the composition comprises DIPA-1-9 at a concentration of 0.05-2.0% wt/vol. In one embodiment, the composition is a liquid composition, and comprises DIPA-1-9 at a concentration of 0.5-20 mg/mL. In one embodiment, the composition is a liquid composition, and comprises DIPA-1-9 at a concentration of 1 to 12 mg/mL. In one embodiment, the composition is a drops and comprises DIPA-1-9 at a concentration of 1-20 mg/mL.

The composition may be provided with suitable packaging and/or in a suitable container. For example, the composition may be in the form of unit dosage unit, for example, a plastic vial, jelly cup, gel, or film strip comprising DIPA-1-9. Alternatively, it can be delivered as a spray.

One aspect of the present discovery pertains to DIPA-1-9 for use in a method of treatment (e.g., targeted treatment) of certain disorders (e.g., a diseases), as described herein. In one embodiment, the medicament comprises DIPA-1-9. In one embodiment, the medicament comprises DIPA-1-9 formulated in a drops and applied with a plastic bottle. Another aspect of the present discovery comprises administering to a patient in need of treatment a therapeutically effective amount of DIPA-1-9, preferably in the form of a pharmaceutical composition.

### Sensory Discomfort and Treatment Objectives

In one embodiment (e.g., of use in methods of therapy, of use in the manufacture of medicaments, of methods of treatment), the treatment is treatment (e.g., selective treatment) of lower airways blocakge disease, such as cough and mucus accumulation. In an another aspect treatment, the DIPA-1-9 is used to stimulate coolness receptors and produce signals that will reduce dyspnea, and alleviate the condition known as sleep apnea. In another aspect of treatment, the DIPA-1-9 is used to facilitate the expectoration of mucus from the airways.

The term “sensory discomfort”, as used herein, relates to irritation, pain, itch, or other form of dysesthesia arising from the lumen of the airways. The term “dysesthesia” as used herein relates to abnormal sensation, and includes, in addition to irritation, itch, and pain, sensations such as burning, dryness, wetness, pins-and-needles, and feeling the presence of a foreign body, and the urge to cough.

In one of the embodiments, the target tissue for DIPA-1-9 is located on an oropharyngeal surface, a hypopharyngeal surface, or a pharyngeal surface. In one of the embodiments, the sensory discomfort from the target tissue is caused by inflammatory exudates in the throat, mucus accumulation, by pharyngitis, by mucositis, by an allergy, by cough, or by hypersensitivity of the pharyngeal surface to an irritant.

In one of the embodiments, the target tissue for DIPA-1-9 is located on an esophageal surface and the sensory discomfort located on an esophageal surface is caused by reflux of stomach contents (e.g., gastroesophageal reflux) or by esophagitis. In one embodiment, the upper aerodigestive tract discomfort is caused by inflammatory exudates in the airways or the pharynx (e.g., associated with asthma, and/or an obstructive pulmonary disorder). In one embodiment, the airway tract discomfort is associated with labored breathing, dyspnea, snoring, or sleep apnea. In one embodiment, the treatment is treatment of oropharyngeal discomfort.

In one embodiment, the treatment is treatment of esophageal discomfort. In one embodiment, the treatment is of throat irritation. In one embodiment, the treatment is treatment of cough or the urge to cough.

In one of the embodiments, the target tissue for DIPA-1-9 is located on an nasal cavity surface, in the context of a lower airway disorder. In one of the embodiments, the sensory discomfort from the target tissue is caused by disturbances in the sensations of breathing. The objective of treatment is to make the subject feel that he or she is breathing clean mountain air, on a clear, cool, and crisp morning.

The term “treatment,” as used herein in the context of treating a disorder, pertains generally to treatment of a human or an animal (e.g., in veterinary applications), in which some desired therapeutic effect is achieved, for example, the inhibition of the progress of the disorder, and includes a reduction in the rate of progress, a halt in the rate of progress, alleviation of symptoms of the disorder, amelioration of the disorder, and cure of the disorder. Inclusive of such treatments are reduction of sensitivity, of hypersensitization, and desensitization phenomena. Treatment as a prophylactic measure (i.e., prophylaxis) is also included. For example, use with patients who have not yet developed the disorder, but who are at risk of developing the disorder, is encompassed by the term “treatment.”

The term “selective” in pharmacological terminology pertains to a molecule that, among a group of structurally related congeners, exhibits unusual qualitative properties that distinguishes it from the other analogs. For example, DIPA1-9 does not have a strong metallic taste, but this taste is present in DIPA-1-7 and DIPA-1-8 and other analogs. Thus, DIPA-1-9 is more selective in its pharmacological actions.

Another aspect of the selective properties of DIPA-1-9 is the low degree of “cold discomfort” compared to the related analogs. DIPA-1-9 can act on surfaces without problems of stinging, irritancy, and pain in the throat or excessive cold behind the sternum.

The “specificity” of the DIPA-1-9 action relates to its efficacy for producing the desired Häagen-Dazs cooling effect. Although all the active analogs are active on the TRPM8 receptor, only DIPA-1-9 fully produces the optimal cooling sensation. Hence, it is more specific for the desired drug action.

The term “therapeutically-effective amount,” as used herein, pertains to that amount of a compound, or a material, composition or dosage form comprising a compound, which is effective for producing some desired therapeutic effect, commensurate with a reasonable benefit/risk ratio, when administered in accordance with a desired treatment regimen.

### Routes of Administration

The pharmaceutical composition comprising DIPA-1-9 may suitably be administered to a subject topically, for example, as described herein. The term “topical application”, as used herein, refers to delivery onto the lumenal surfaces of the nasal cavity, pharyngx and esophagus.

The subject/patient may be a mammal, for example, a marsupial (e.g., kangaroo, wombat), a rodent (e.g., a guinea pig, a hamster, a rat, a mouse), murine (e.g., a mouse), a lagomorph (e.g., a rabbit), avian (e.g., a bird), canine (e.g., a dog), feline (e.g., a cat), equine (e.g., a horse), porcine (e.g., a pig), ovine (e.g., a sheep), bovine (e.g., a cow), a primate, simian (e.g., a monkey or ape), a monkey (e.g., marmoset, baboon), an ape (e.g., gorilla, chimpanzee, orangutan, gibbon), or a human. In one preferred embodiment, the subject/patient is a human.

### Formulations for Delivery

The preferred formulation of DIPA-1-9 is to dissolve it in liquid drops using water, a water-based solution, or syrup. Other ingredients that may be included are fillers, buffers, preservatives, anti-oxidants, lubricants, stabilisers, masking agents, coloring agents, and flavoring agents. The formulation may further comprise other active pharmacological agents. If formulated as discrete units (e.g., vials, pre-wrapped units), each unit contains a predetermined amount (dosage) of the compound.

The term “pharmaceutically acceptable,” as used herein, pertains to compounds, ingredients, materials, compositions, dosage forms, etc., which are, within the scope of sound medical judgment, suitable for use in contact with the tissues of the subject in question (e.g., human) without excessive toxicity, irritation, allergic response, or other problem or complication, commensurate with a reasonable benefit/risk ratio. Each carrier, diluent, excipient, etc. must also be “acceptable” in the sense of being compatible with the other ingredients of the formulation.

Suitable carriers, diluents, excipients, etc. can be found in standard pharmaceutical texts, for example, Remington's Pharmaceutical Sciences, 18th edition, Mack Publishing Company, Easton, Pa., 1990; and Handbook of Pharmaceutical Excipients, 5th edition, 2005. The formulations may be prepared by any methods well known in the art of pharmacy. Such methods include the step of bringing into association the compound with a carrier which constitutes one or more accessory ingredients. In general, the formulations are prepared by uniformly and intimately bringing into association the compound with carriers (e.g., liquid carriers).

### Dosage

It will be appreciated by one of skill in the art that appropriate dosages of DIPA-1-9, and compositions comprising DIPA-1-9, can vary from patient to patient. Determining the optimal dosage will generally involve the balancing of the level of therapeutic benefit against any risk or deleterious side effects. The selected dosage level will depend on a variety of factors including, but not limited to, the activity of DIPA-1-9, the route of administration, the time of administration, the duration of the treatment, other drugs, compounds, and/or materials used in combination, the severity of the disorder, and the species, sex, age, weight, condition, general health, and prior medical history of the patient. The amount of DIPA-1-9 and route of administration will ultimately be at the discretion of the physician, veterinarian, or clinician, although generally the dosage will be selected to achieve local concentrations at the site of action which achieve the desired effect without causing substantial harmful or deleterious side-effects.

Administration can be effected in one dose, preferably on an “as-need” or pro re nata basis throughout the course of treatment. Methods of determining the most effective means and dosage of administration are well known to those of skill in the art and will vary with the formulation used for therapy, the purpose of the therapy, the target receptors being treated, and the subject being treated. Single or multiple administrations can be carried out with the dose level and pattern being selected by the patient, treating physician, veterinarian, or clinician.

### Airways Physiology

Air enters the nasal cavity and goes to the trachea and then down the bronchioles to the gas exchange surfaces of the alveoli. Food, air, and liquids enter the mouth, goes past the pharynx, and into the esophagus and stomach. The nerve endings around the epiglottis and glottis are especially sensitive to mechanical perturbations. The tube that forms the upper airways is distinct from the lower airways which begins beneath the glottis. In this application, DIPA-1-9 drops are administered to the upper airways to treat disorders of the lower airways. This concept is a source of novelty.

The oral cavity contains specialized structures such as teeth, gums, tongue, and salivary glands that are designed to masticate, taste, lubricate, and propel the food bolus into the pharynx. This is a complicated muscular reflex activity that requires the coordination 6 cranial nerves and 25 muscle groups. Heat sensation is not a high ranking protective reflex in the oral cavity as the mouth can tolerate hot liquids which are painful when put on the skin. Cooling liquids, on the other hand, are important in the regulation of thirst. Eccles et al. [Cold pleasure. Why we like ice drinks, ice-lollies and ice cream. Appetite, 71, 357-60, 2013] reviewed and emphasized this concept on the relationships of cooling liquids, ice creams, positive reinforcement, and the suppression of thirst. Sensory nerves closely monitor temperatures at the junction of the oral cavity and pharynx. When the external ambient temperature is high, drinking cooling liquids is instantly pleasurable and relieves thirst, dryness, and discomfort.

The pharynx is a passageway leading from the nasal and oral cavities to the larynx and esophagus. The pharynx is part of the throat, an inexact term describing the region of the body around the neck and voice-box. The pharynx is divided into three regions: naso-, oro- and laryngo-. The nasopharynx, also called the rhinopharynx, lies behind the choanae of the nasal cavity and above the level of the soft palate. The oropharynx reaches from the soft palate (velopharynx) to the level of the hyoid bone. The laryngopharynx (also called hypopharynx) reaches from the hyoid bone to the lower border of the cricoid cartilage. The pharyngeal and esophageal surfaces are lined with stratified epithelium. By contrast, the respiratory epithelia of the nasopharynx, larynx and trachea are a single layer of cells, usually with cilia. The transition from stratified to respiratory epithelia occur at the base of the epiglottis.

The oropharynx may be further divided into an upper and lower region, the mid-point being what is called the lower retropalatal oropharynx (LRO) as shown, for example, in the magnetic resonance imaging studies of Daniel et al. [Pharyngeal dimensions in men and women”, Clinics (SaoPaulo) 62, 5-10, 2007]. The pharynx is a trapezoid inverted funnel-shaped tube and the LRO is the region with smallest cross-section, an area of about 1 cm², which is equivalent to 20% of US quarter coin of 25% of a Euro coin. The pharyngeal surface at the base of the tongue and the pharyngeal wall around the LRO, with a total area of about 3 to 5 cm², is a key part of the desired target for drug delivery for the methods described herein.

The traffic that passes through the lumen of the oropharynx every day is astounding. On an average day, an adult breathes 12,000 L of air, drinks 2 L of fluids, secretes 1 L of saliva, and eats 2 kg of food. These activities are constant, with about 15 breaths and 1 swallowing movement per min during the waking hours. For the organism to survive, the traffic flow must be co-ordinated so that food and liquids go down the esophagus and not into the airways, and air gets directed into the airways. Hence, this surface is densely innervated with sensors in the form of nerve endings of the 9^{th} and 10^{th} cranial nerves.

The brain co-ordinates pharyngeal traffic via striated and smooth muscle effectors, For solids, the food is masticated, mixed and lubricated with saliva, and the bolus is then rapidly pushed down to the esophagus. The oropharyngeal phase of swallowing occurs in the blink of an eye, in millisec, as the bolus transits down the pharynx at about 35 cm/sec. The pharyngeal transit time of a bolus past the mandible, then past the esophageal sphincter is less than 1 sec. Some of the sensory signals that govern this process in the mouth and rostral tongue come from afferents of the trigeminal nerve (5^{th}) and hypoglossal nerve (8^{th}). The afferent signals from the oropharynx and posterior surface of the tongue come mainly via glossopharyngeal nerve (9^{th}). Signals from the laryngopharynx (also called the hypopharynx) are mainly via the vagus nerve (10^{th}). Swallowing and coughing are reflexes designed to direct traffic load to their correct destinations.

The neuronal receptive fields of the nerve endings in the oropharyngeal epithelia are sub-served by the 9^{th} and 10^{th} cranial nerves. These nerve endings are the targets of DIPA-1-9 as shown in FIG. 2. The pharyngeal surface cells have a high turnover rate (on the order of several days) and are sensitive to injury. For example, when there is disorganized traffic of solids or liquids in the pharynx, or when acid and pepsin, or exudates from the lungs, accumulate, the upper airway tract will activate the cranial nerves and convey signals of irritation, itch, pain, and the urge to cough. The characteristic manifestations of pharyngeal disorders are globus (the feeling of a lump in the throat), difficulties in swallowing (dysphagia), difficulty in breathing (dyspnea), hoarseness, pain, itch, cough, and redness and swelling of the pharyngeal mucosa. Impairment of airflow by malfunction of the airways is also associated with acute anxiety and a sense of impending doom.

FIG. 2. is a drawing of the innervation of the human pharynx, demonstrated by the Sihler's stain. A target for placement of the DIPA-1-9 drops is shown in the black-outlined circle, at the base of the tongue. The drawing is adapted from Mu and Sanders, “Sensory nerve supply of the human oro- and laryngopharynx: a preliminary study.” Anatomical Record 258:480-420, 2000. The nerve endings of the upper oropharynx are primarily from the 9^{th} nerve (glossopharyngeus), and the nerve endings for the laryngopharynx, near the glottis, from the 10^{th} nerve (vagus). The lateral and posterior walls of the oropharynx are innervated by both the 9^{th} and 10^{th} nerves. Epi=epiglottis, medium black areas =tonsils, and the small black areas are lymph granules and taste buds. The nerve endings transduce the signals from the pharynx to the brain and coordinate sensory perception and muscular response.

The pharynx has strong, constrictor muscles, arranged as a vice and designed to grab the oropharyngeal contents and push the bolus into the esophagus. The anatomy is like the first baseman glove in baseball. There are two important valves in this system: the epiglottis which closes during swallowing, and the upper esophageal sphincter (UES, or cricopharyngeus muscle) which relaxes to allow the contents to enter the esophagus, then shuts to prevent reflux. Pharyngeal contractions flush and empty the lumen of debris, and by creating negative pressure helps suck contents from the nasal cavity and nasopharynx. Well-toned pharyngeal muscles are important for maintaining patency of the airways, allowing smooth airflow and dysfunction will cause dysphagia, dyspnea, snoring, and sleep apnea. Swallowing is also important for removal of mucus wafted up the trachea via ciliary clearance.

Examples of airway disorders in which a topical DIPA-1-9 drops exerting an ideal cool sensation may have utility are:

### Chronic Lower Airways (Lung or Pulmonary) Blockage Disorders

This condition is the primary focus of this application. The airways and the lungs are like the heart, brain, liver, and kidneys—a major organ system essential for survival. Damage to the airways are quite common, and an extensive cause of human suffering, morbidity, and mortality. For example, chronic obstructive pulmonary disease is the third to fifth leading cause of death in most of the countries in the world.

In lower airway disorders, inflammation or damage of the airway walls increases all secretions, including hypersecretion of mucus. The phelgm, which is normally cleared by mucociliary action, is now wafted in abundance, up the bronchi, into the trachea, then into the glottis. The phlegm chokes the airways. The clinical results of the lower airway blockage disorders are cough, increased sputum production, and dyspnea (in more severe cases because of blockage), and the difficulties in breathing sensations may cause anxiety and panic attacks. Control of cough discomfort relieves patient anxiety and enables the deliberate clearance of accumulated mucus by the patient. With psychic control of throat discomfort the patient has less anxiety and gets a good night's sleep.

Cough. Cough (and the urge to cough) is a common experience. Cough stimuli can enter the throat by inspiration, e.g. smoke and nasal secretions, or be expired into the throat from the airways, e.g. phlegm. Each cough involves co-ordinated muscular effort of inspiration, compression and expiration. An effective cough can generate air velocity of 280 m/sec and volumes of 12 L/sec. Cough clears the airways of secretions and particles. But, it can also be non-productive (dry and hacking), painful to the throat, and exhausting because of increased muscular effort. The throat lining can become hypersensitive to innocuous stimuli. If patients are taught to control cough they can sleep better at night and this control may be utilized to clear mucus. The preferred embodiment of the active ingredient DIPA-1-9 rapidly cools the throat surface via activation of TRPM8 receptors. The sensation evoked is like swallowing a spoonful of a rich ice cream, but lasts longer. DIPA1-9 is administered formulated in liquid form with water or syrup and administered as “drops” onto the back of the throat on an “as needed basis [pro re nata]”. In less than 2 min, the urge to cough is suppressed. The dose can be repeated to control the urge to cough. The drops also works on sore throat and indigestion.

Dyspnea is a common symptom of airway disease and is defined as “a sensation of difficult breathing” which includes sensations of breathlessness, choking and suffocation. As a sign, severe dyspnea is expressed as labored breathing and inadequate ventilation with a rise in plasma carbon dioxide tension. Dyspnea occurs in serious disorders such as pneumonia, congestive heart failure, asthma, chronic obstructive pulmonary disease, emphysema, cystic fibrosis, muscular paralysis or dystrophy, Parkinson's disease, lung cancer, debilitation from wasting diseases and the like. The sense of suffocation, encompassed in dyspnea, is a frightening experience. For example, in amyotrophic lateral sclerosus, 56% of patients experience dyspnea in the last month of life. Sleep apnea and snoring are also associated with uncomfortable breathing. Surprisingly, cooling of the upper airways with DIPA-1-9 drops can relieve dyspnea. It is likely that cooling sensations from the upper airways convey a sense of fresh airflow. Spence et al. (Chest 103: 693-696, 1993) has shown that COPD patients have a better exercise capacity when breathing cold air.

Mucus Clearance and “Efficient Cough”. Diseases of the airways, such as COPD, bronchitis, bronchiectasis, cystic fibrosis, and certain forms of asthma have increased production of exudates. The mucus hypersecretion is removed by either coughing or swallowing. Coughing is effective in clearing secretions down to the 7^{th} to 12^{th} of the total of 23 airway generations. The velocity of airflow in a cough may be 250 to 280 m/sec, which is close to the speed of sound (343 m/sec). Air compression may go up to 300 mmHg. At night and during sleep, muscles relax and clearance is inhibited, so accumulation cause choking and gagging. A sensory agent that counteracts discomfort in the throat may be used to promote mucus awareness and clearance, without hurting the throat lining via non-productive coughs. The cooling sensation may enhance topographical awareness of the physical dimensions of the mucus droplets in the oropharynx and facilitate swallowing of mucus. This is an additional mechanism for mucus clearance. Therapeutically, mucus clearance is a very important endpoint for treatment. Only cough and swallowing are the pathways for removal of mucus.

Cough Hypersensitivity Syndrome (CHS). CHS was defined by the European Respiratory Society as a condition in which the cough is caused by stimuli that don't usually cause cough, or a hypersensitivity to stimuli that are known to be tussive, e.g. citric acid or capsaicin. While this hypersensitive mechanism has been imputed initially in patients with chronic cough where no cause of the cough has been found, there is now evidence that even in patients with chronic cough associated with conditions such as asthma, chronic obstructive pulmonary disease, pulmonary fibrosis or gastroesophageal reflux disease, this mechanism is underlying the chronic cough. So, patients with CHS may have hypersensitivity to stimuli that do not usually induce coughing e.g. talking, laughing, going outside in cold weather or smelling perfume. Other common complaints are a sensation of having something stuck or irritating in the throat, and difficulty breathing such as a feeling that there is a blockage at the level of the throat and the patient can't get air into the lung. Most patients presenting with a chronic cough have CHS. An agent such DIPA-1-9 in drops which is an anti-irritant and “tones” down the sensitivity of the nerve endings should work for CHS.

Asthma. Is a chronic lower airway disorder of the airway tubes, characterized by inflammation, wheezing, cough, increased airway resistance, and troubled breathing. In some forms of asthma, there is copious mucus secretions. Most people have “allergic asthma”, which means that the disease is triggered by allergens.

Pharyngitis: An inflammation of the pharyngeal lining which is most commonly caused by viral and bacterial agents, and by inflammatory exudates that come up the airways. A closely related condition is tonsillitis [Bathala, S. and Eccles, R. A review on the mechanism of sore throat in tonsillitis. Journal of Laryngology and Otology, 127: 227-32, 2013]. Chemical pollutants, such as cigarette smoke, can also directly irritate and damage the mucosa. The principal symptoms of pharyngitis and tonsillitis are irritation, itch, and pain or a “sore throat”. Prolonged pharyngeal irritation can also lead to a chronic hypersensitivity syndrome manifested by persistent cough (called chronic cough when it is present for ≥8 weeks). The DIPA-1-9 formulation described herein will relieve the discomfort of chronic pharyngitis and cough.

Post-nasal drip (upper airway cough syndrome): A condition where increased secretions enter the orpharynx from the mucosa of the nasal cavities and nasopharynx. These secretions may contain inflammatory exudates and may arise from infections or allergy of nasal and sinonasal membranes (for example, allergic rhinitis, and rhinosinusitis). The increased secretions cause throat discomfort, pain, itch, the urge to cough, and a sense of impaired airflow.

Laryngopharyngeal reflux disease (LPR) and esophageal reflux disease: In LPR, stomach acid and pepsin are regurgitated onto the laryngopharyngeal surfaces and causes tissue injury. Normally, proper deglutition and a constricted upper oesophageal sphincter (UES), prevent regurgitation, but when this system is impaired, the acid and pepsin enters the pharyngeal surfaces and can even enter the Eustachian tubes and the nasal sinuses. The result is a syndrome of hoarseness, pain, laryngoedema, and persistent throat clearing. Examination of the larynx shows red and swollen mucosae about the voicebox. A agent that reduces discomfort is likely to be useful in the treatment for LPR. Currently, the primary method of treatment is to reduce acid secretion from the stomach, for example, with the use of proton-pump inhibitors; however, there are no methods to treat the discomfort in the throat. An agent such as DIPA-1-9, formulated for delivery in a drops offers a novel strategy for therapy of reflux disease. Strictly speaking, an acid reflux disorder causing airway irritation may occur on both the upper and lower airways to trigger discomfort.

In the context of the present discovery, the goals were to:
a) Identify and define an active compound(s) with a precise sensory effect on the membranes of the upper airways that will produce a stimulus of coolness and counteract discomfort (irritation, itch, and pain). This sensation generates a feeling similar to when rich ice cream is swallowed but lasts longer. In choosing an active compound(s), a sensations to avoid are conditions referred to as “icy cold” or “cold discomfort”.
b) Develop a topical formulation for localized delivery of the active compound onto targets of the nerve endings of the 9^{th} and 10^{th} cranial nerves. This was accomplished using a reservoir bottle to deliver drops of a liquid solution of the active ingredient in a water, water-based liquid, or a syrup-based liquid. The drops were each approximately the volume of a minim (0.05 mL).
c) Define a drug action with rapid onset (less than 2 min) and long duration (effective for at least several hours), with a dosage schedule that can be based on an “as needed” basis (pro re nata or p.r.n.), and thus allowing the patient to regain control of the sensory discomfort such an itch or an urge to cough. Ideally, the active compound is sufficiently potent, with a unit dose of about 0.5 to 5 mg per unit dose of administration.
d) Use this medication for short-term (acute) and long-term (chronic) conditions, such as cough, mucus clearance, and to control discomfort and to reduce hypersensitivity to irritant stimuli.

These objectives are met with a drop formulation of DIPA-1-9 at a delivered volume of less than 0.5 mL per total dosage with focused delivery onto the nerve endings of the 9^{th} and 10^{th} nerves, that is, at the base of the tongue, on the border of the oral cavity and the oropharynx, and on the lateral walls of the oropharynx.

Targeted Topical Delivery onto a Specific Location

To create a drug for topical delivery to the pharyngeal and esophageal surfaces requires understanding of target tissues and dynamics of the tissue environment. The neuronal receptive fields of the pharynx and esophagus are linked to the afferents of the 9^{th} [glossopharyngeal], 10^{th} [vagus], and spinal afferents. The area for delivery is about several cm². The area of the oral cavity is at least 10× larger. So, a chewing gum containing DIPA-1-9 will not work well, because most of the active ingredient will go onto the surface of the oral cavity. A teaspoon of DIPA-1-9 in drops will not work either because the volume of 5 mL in a teaspoon is too large and the contents will transit through the pharynx via swallowing and pass into the esophagus before the DIPA-1-9 has opportunity to interact with its receptor. The drops are ideal, but a rapidly dissolving oral tablet or a film placed in the back of mouth are possible alternatives.

The oropharynx at its entrance is an arch-shaped structure at the base of the tongue, with the uvulva [or grape] hanging in the middle. The base of the arches, called the anterior pillars of fauces, is especially sensitive to cold sensations. If a cold metal probe is placed at this site in human subjects, cooling sensations and rapid swallowing movements are elicited [Kaatzke-McDonald, E. et al. The Effects of Cold, Touch, and Chemical Stimulation of the Anterior Faucial Pillar on Human Swallowing. Dysphagia 11:198-206, 1996]. The pharyngeal surfaces are densely innervated by nerve endings of 9^{th} and 10^{th} cranial nerves (FIG. 2). TRPM8 immunoreactive fibers are abundant at the border of the oropharynx but not in the epiglottis [Sato, T. et al. The distribution of transient receptor potential melastatin-8 in the rat soft palate, epiglottis, and pharynx. Cellular and Molecular Neurobiology, 33:161-5, 2013]. The desired drug targets for treatment of the lower airway disorders are on the receptive fields of 9^{th} and 10^{th} nerve, and for dyspnea, on the 5^{th} nerve. The drug targets are in the upper airways.

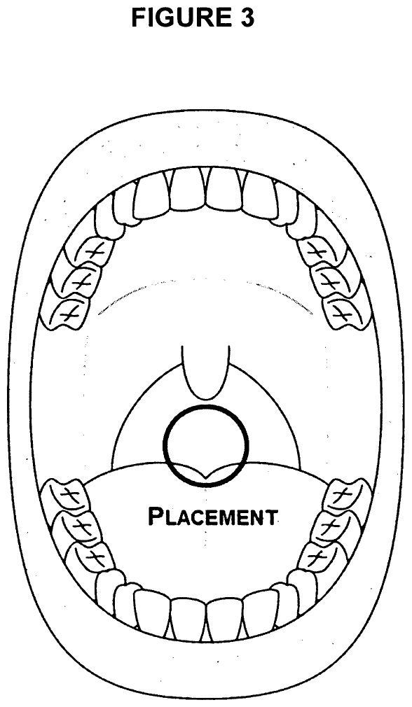

The favored target for drug delivery is the lumenal surfaces of the oropharynx at the base of the tongue, next to the pillars of fauces, and also further back for delivery to the lateral oropharyngeal walls (see FIG. 3). A secondary target is the lumen of the upper esophagus which is reached via the oropharynx. A third target for treatment of dyspnea is the lumen of the nasal cavity.

The afferent signals to the brainstem from the oropharynx and the laryngopharynx are primarily from the 9^{th} (glossopharyngeal) and 10^{th} (vagus) cranial nerves. The afferent signals from the receptive fields coordinate the clearance reflexes that empty the pharynx and protect the airways against entry of liquids and solids. For the upper esophagus, the innervation is from the vagus and spinal afferents. The targets for drug delivery are primarily the receptive fields of the 9^{th} and 10^{th} cranial nerves, and, to a lesser extent, the spinal afferents of the upper esophagus.

The oropharyngeal phase of swallowing occurs in the blink of an eye, in millisec, as the bolus moves from mouth to esophagus. The transit time, as measured by laser Doppler ultrasound or X-ray videofluorography is about 35 cm/sec [Sonomura et al., Numerical simulation of the swallowing of liquid bolus. J. Texture Studies 42: 203-211, 2011]. Regueiro et al. (Influence of Body Height on Oral and Pharyngeal Transit Time of a Liquid Bolus in Healthy Volunteers. Gastroenterol Res. 2018; 11(6):411-415) measured a transit time of ˜0.5 sec for the passage of a bolus from the mandible past the esophageal sphincter. So it is therefore difficult to deliver, adhere, glue, and retain a sensory agent on the surface of the oro-laryngopharynx. The active ingredient cannot be delivered as solid particles, as that would cause irritation and elicit coughing, so delivery of a agent in liquid drops is the ideal method. An agent in a thickened spray may also work, but a highly aerosolized spray will activate airway receptors in respiratory epithelia and cause cough. An agent present as a solute in saliva is diluted in the mouth and still has to be retained in sufficient concentration to contact the pharyngeal surface. A liquid drop does not depend on secretion of saliva.

### Onset, Duration of Action, and Schedule of Delivery

As contemplated here, the delivered agent for treatment should have a sensory effect with rapid onset of action, for example, within 2 min. The effects should be effective for at least one hour and preferably longer, otherwise the patient would have to repeatedly apply the drug to obtain relief. Preferably, there should be a “wow effect” of the active ingredient to stimulate sensory events. The patient should be able to identify this “wow effect” and use the liquid formulation on an “as needed” (p.r.n.) basis. With a fast onset of action, the patient should be able to be relieved of oropharyngeal discomfort, and this relief will further reduce psychogenic factors (e.g., anxiety) associated with throat discomfort. These goals are achieved by DIPA-1-9 dissolved in a small volume of water or drops and applied to the base of the tongue. The success of this treatment is also based on “instant gratification”, namely, the immediate relief of discomfort.

### Choice of Active Ingredient: Molecular Target, Specificity, Selectivity

There is a general view that the ion channel TRPM8 is the principal physiological element that transduces to the brain the cooling effects of agents such as menthol and icilin [McKemy et al., Identification of a cold receptor reveals a general role for Trp channels in thermosensation, Nature, 416, 52-58, 2002]. TRPM8 is an integral membrane protein with 1104-amino acid residues and has six transmembrane domains. Activation of this receptor by decreasing ambient temperature results in the opening of a gate in the TM (transmembrane) 5-6 loops and non-specific cation entry into the cell. The entry of cations depolarize sensory neurons and the action potentials are transmitted to the brain primarily via Aδ (and some C) fibres. This is a transduction system. Whilst this concept for the role of TRPM8 in sensory physiology may be valid for physical changes in temperature, the interpretation of the sensory effects of chemical agents such as menthol and icilin are more complex. Menthol not only stimulates TRPM8 in vitro, but also TRPV3, a receptor associated with warmth [Macpherson et al., More than cool: promiscuous relationships of menthol and other sensory compounds. Mol Cell Neurosci 2006; 32:335-343, 2006]. Menthol also inhibits TRPA1. Menthol is “non-selective” in its actions. Icilin stimulates not only TRPM8, but also TRPA1, and icilin inhibits TRPV3 [Sherkheli et al., Supercooling agent icilin blocks a warmth-sensing ion channel TrpV3. Scientific World Journal 2012; 982725, 2012] and glycinergic transmission [Cho et al. TRPA1-like channels enhance glycinergic transmission in medullary dorsal horn neurons. J Neurochem 122:691-701.2012]. Thus, menthol and icilin are “promiscuous” non-selective drugs and their actions may not be associated with any one particular receptor protein. Hence, the sensory effects of menthol and icilin are difficult to categorize as “pure-TRPM8” or something else.

The correlation between a chemical's potency at the TRPM8 receptor (measured by the median effective concentration or EC₅₀) and ability to evoke sensory events in the pharynx is complex. The applicant has examined compounds covering a 100-fold range of TRPM8 potency, each of which exhibited full efficacy at the TRPM8 receptor, and evaluated their sensory effects. A number of side-effects were observed with some of the compounds. For example, pure menthol crystals produced chest discomfort at a dose of 5 mg in an orally dissolving tablet. By contrast, icilin did not produce cooling in the chest or the desired sensations on the throat. Among the dialkylphosphorylalkane (DAPA) compounds, the relationships of TRPM8 receptor potency to sensory events were not easily separated. Firstly, the DAPA compounds with 6 to 8 methyl groups in the longest alkyl chain have aversive tastes in the oral cavity. Secondly, these 6 to 8 analogs caused icy cold pain and discomfort in the laryngx and behind the sternum. Surprisingly, DIPA-1-9, has all of the desirable qualities for an ideal cooling agent on the oropharynx, even though it is not “super-potent” on TRPM8.

As shown in Study 4, the EC₅₀ [median effective dose] of a candidate for activating TRPM8 has little predictive value in identifying a candidate for treatment of sensory discomfort in the upper aerodigestive tract. This is not surprising and to over-interpret the EC₅₀ value is naïve. The 95% Confidence Limits of many EC₅₀ values overlap and are not easily differentiated from each other. The EC₅₀ values do not give information on the quality of the heat abstraction sensation, the duration of action, or the likelihood of unpleasant taste. Thus, identification of selective agents requires multiple bioassays and an optimized delivery system.

When it became clear that TRPM8 receptor potency screening could not be used as the primary method for selection of an active ingredient, it was necessary to precisely define the distinct sensations of a test compound applied to the oropharyngeal surface. These descriptors are summarised in Table 4. For any compound, there may be some overlap in activity, but usually one compound occupies only one or two categories of sensations (FIG. 5).

Cooling agents have different qualities in oral cavity and oropharynx. In the mouth, the gradations of cold are limited and are described as neutral, cold, or icy. In the throat, however, one can distinguish among the finer gradations of cool, refreshing cool, cold, and icy cold. Using the appropriate stimulus such as rich ice cream, sherbet, and super-icy lemonade, these distinct levels are recognized as part of everyday experience. The varied sensitivity in the oropharynx is due to the dense topographical neuronal receptive fields. As the bolus transits downwards, the information transduced from the oropharyngeal surface are both the dynamic and static temperatures: that is, the brain “feels” −Δ° C./t and not just absolute ° C. The mouth just only “feels” the static temperature. Only an agent that simulates optimal Δ° C./t on nerve discharge will produce “refreshing cooling”.

Coolness also enhances the sensory “awareness” of what is in the oropharynx. So the presence, size, and shape or any bolus, liquid or solid, is “sized” and this information is sent to the brain for execution of tasks, such as swallowing or coughing. This is an important parameter for coughing and mucus clearance.

TABLE 4
Description of sensations and comments.

| Oropharynx | Descriptor | Heat abstraction sensations, analogy |
|---|---|---|
| Inactive | No effect | — |
| Cool | Cool | Drinking room temperature water |
| Ideal cool | Ideal cool | rich ice cream, such as Häagen-Dazs ice cream, |
| | | smooth |
| Cold | Cold | Sherbert, can be numbing |
| Icy cold | Icy cold | Icy lemonade, painful, chest discomfort |

In the upper airways, DIPA-1-9 elicits cool by action on receptive fields of afferents located in the pharynx. The sensory nerves present include the facial (7^{th})-innervating the surfaces adjacent to the palatine tonsils, the glossopharyngeal (9^{th})-innervating the posterior of the tongue and walls of the oropharynx, and the vagus (10^{th})-innervating portions of the lateral/posterior walls of the oropharynx and the laryngopharynx. Further down the digestive tract, the upper esophagus is innerved by the vagus and spinal afferents. The distributions of these nerve fibers in pharynx are shown in FIG. 2 and constitute the targets for drug action. For dyspnea of COPD, the targets are on the nerve endings of the nasal cavity, which is innervated by the 5^{th} nerve.

Technical difficulties prevent direct measurement of sensory inputs from the receptive fields of the 7^{th}, 9^{th} and 10^{th} nerves, but mapping has been done for the 5^{th} nerve, from receptive fields of the snout skin of rats. By inference, one can presume the processing of information is the same for all of these cranial nerves. The central response of the 5^{th} nerve neurons has been recorded and studied from rat superficial medullar dorsal horn that responds to innocuous thermal stimulation of the rat's face and tongue. Step changes of −Δ2.5 to 5.0° C. stimulated cells with both static firing rates and cells with dynamic properties [Davies, S N et al. Sensory processing in a thermal afferent pathway. J. Neurophysiol. 53: 429-434, 1985]. Similar studies in cats and humans showed that step decreases in temperatures (dynamic changes), as low as −Δ0.5° C./sec, were readily detectable by neurons and by psychophysical measurements [Davies, S N et al. Facial sensitivity to rates of temperature change: neurophysiological and psychophysical evidence from cats and humans. J. Physiol. 344: 161-175,1983]. From a study of the spike patterns of neuronal discharge (impulses/sec), it was clear that dynamic and not static firing rates were the most powerful stimuli for generating coolness/cold sensations [Davies et al. 1983]. That is, the brain “sees” −Δ° C./t and not absolute ° C. Thus, an agent that simulates optimal −Δ° C./t on nerve discharge will produce “ideal cool”. Thus, one can see why cool temperature sensing in the static conditions of the oral cavity are different from the sensations felt in the lumen of the nasal cavity, pharynx and esophagus as the bolus or drops pass through.

### Delivery to Target: Place and Selecting the Right Concentration

In this invention, one of the goal is to apply DIPA-1-9 in a small volume drops onto the receptive fields of the 9^{th}, and 10^{th} cranial nerves to counteract irritation, itch, and/or pain in the pharynx, and those noxious signals arising from the lower airways. The fast transit time (<1.0 sec) of solids/liquids through the oropharynx is a hindrance to topical drug delivery to the receptive fields, but this obstacle can be circumvented by formulation of the active ingredient into a milieu that adheres to the target. This is especially achieved when DIPA-1-9 is delivered as drops formulated in water or syrup.

The DIPA-1-9 having liquid miscibility and chemical stability, is ideal for delivery as a focused liquid aliquot (drops) to a desired location. For example, drops may be convenient for individuals who are unable easily to use solid dosage forms, e.g. young children, the elderly, and disabled individuals with difficulties in salivating or swallowing. By using drops liquid delivery is uniformly dispersed and adheres with sufficient contact time on pharynx and avoids rapid transport down into esophagus. Unlike a lozenge, delivery of drops to target does not depend of secretion of saliva.

A preferred formulation is a DIPA-1-9 formulation in water or a syrup at a concentration of 2 to 10 mg/mL and administered as single unit aliquots of 0.2 to 1.0 mL onto the base of the tongue. Such a formulation exerts a sensory effect in less than 2 min and is effective for several hours for throat discomfort and heartburn. A preferred liquid formulation is 5 mg/mL of DIPA-1-9 dissolved in water. If syrup is used, it can be purchased ready made from Humco Compounding, Austin, Tex. These solutions can be placed in a plastic vial with a nozzle tip and administered to the back of the mouth. Alternatively, the drops may be place in a reservoir bottle with a manually activated spray pump with a spacer attachment of 3 inches (˜7.5 cm) that will facilitate delivery onto the surfaces at the back of the mouth. Another possible formulation is the use of quick-dissolving liquid gel or film that can be placed in the back of the mouth, at the base of the tongue.

The schedule of delivery of the agent is designed for an “as-needed” basis by the patient, and does not require a fixed-interval. By this therapeutic strategy, the individual has voluntary control of upper airways discomfort, and can, for example, sleep better at night, gain peace of mind, and have less anxiety. Alternatively, in the treatment of the cough hypersensitivity syndrome, when the objective is to reduce neuronal hypersensitivity, a fixed interval regimen may work better.

### Study 1

Agonist Potency and Selectivity on TRP channels: TRPM8, TRPV1, and TRPA1

In the frist set of data, the potency and in vitro effects of test compounds were evaluated on cloned hTRPM8 channel (encoded by the human TRPM8 gene, expressed in CHO cells) using a Fluo-8 calcium kit and a Fluorescence Imaging Plate Reader (FLIPR^{TETRA}™) instrument. The assays were conducted by ChanTest Corporation (Cleveland, Ohio 44128, USA). Test solutions were in a HEPES-buffered saline, in 384-well plates, and placed into the FLIPR instrument (Molecular Devices Corporation, Union City, Calif., USA). Four 4 to 8 concentrations were tested, with L-menthol as the positive control. The test cells were Chinese Hamster Ovary (CHO) cells stably transfected with human TRPM8 cDNAs. Concentration-response data were analyzed via FLIPR Control software) and fitted to a Hill equation for the EC₅₀. The 95% Confidence Interval was obtained using GraphPad Prism 6 software.

The results (agonist activity in the TRPM8 receptor assay) are summarized in Table 5. All tested compounds showed full efficacy, i.e. at the highest tested concentration there was ˜100% stimulation of calcium entry, and the data fitted a sigmoidal dose-response curve. The EC₅₀ of the more potent sensory compounds DIPA-1-6 to 1-9, and DIPA-2-5 to 2-8 fell within a narrow range with overlapping 95% Confidence Intervals. There were no distinguishing features in the EC₅₀ which enabled prediction of the compounds with desired cooling properties in the upper airways. The structural modifications of 3-1 and 3-2 resulted in a significant loss of bioactivity.

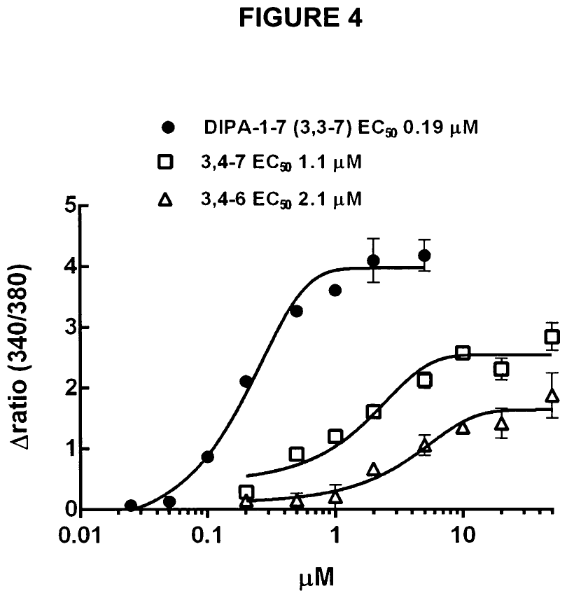

In a second set, tests were made on “mixed” isopropyl-sec-butylphosphorylhexane and heptane analogs described as 3,4-6 and 3,4-7 in Table 2, and results shown in FIG. 4. The data were collected by Andersson et al. of King's College, London, UK, using his methods described in “Modulation of the cold-activated channel TRPM8 by lysophospholipids and polyunsaturated fatty acids. Journal Neuroscience 27 (12): 3347-3355, 2007. Here, the cellular entry of the calcium-sensitive dye Fura-2 was used to study the effect of the test compounds on TRPM8 expressed in Chinese hamster ovary cells. Cells, grown in culture, were seeded at an approximate density of 30,000 cells/well overnight, and loaded for ˜1 hr with 2 M Fura-2 (Molecular Probes, Leiden, The Netherlands), and then placed on glass coverslips. Test solutions were added with a micropipette positioned close to the cells. Emission intensity from cells was measured for 90 sec, at every 4 or 5 sec, using excitation wavelengths of 340 and 380 nm and an emission of 520 nm. Fluorescence emission intensity ratios at 340 nm/380 nm excitation (R, in individual cells) were recorded with a FlexStation and the ImageMaster suite of software (PTI, South Brunswick, N.J.). Samples were tested in triplicate at each concentration and the averaged values analyzed by non-linear regression using an a sigmoidal function fit of the points to obtain an estimated EC50 (median effective concentration) (GraphPad Prism software, La Jolla, Calif.).

TABLE 5
TRPM8 agonist activity of test compounds.

| | | 95% Confidence | Relative Potency to |
|---|---|---|---|
| Compound | EC₅₀ (μM) | Interval | L-menthol |
| Menthol | 3.8 | 2.5 to 5.6 | 1.0 |
| DIPA-1-5 | 5.6 | 4.4 to 7.2 | 0.7 |
| DIPA-1-6 | 2.4 | 1.5 to 4.0 | 1.6 |
| DIPA-1-7 | 0.7 | 0.5 to 1.0 | 5.4 |
| DIPA-1-8 | 0.7 | 0.5 to 1.0 | 5.4 |
| DIPA-1-9 | 0.9 | 0.4 TO 2.5 | 4.0 |
| DAPA-2-4 | 14.5 | 7 to 29 | 0.3 |
| DAPA-2-5 | 1.7 | 1.0 to 2.9 | 2.2 |
| DAPA-2-6 | 0.8 | 0.5 to 1.3 | 4.7 |
| DAPA-2-7 | 1.1 | 0.6 to 2.3 | 3.4 |
| DAPA-2-8 | 1.3 | 0.7 to 2.3 | 2.9 |
| DAPA-3-1 | 24 | 8 to 76 | 0.2 |
| DAPA-3-2 | 4.2 | 1.6 to 10.8 | 0.9 |

The potency of three analogs for activation of TRPM8 (cooling receptor) in transfected cells is shown in FIG. 4. The units (Δ ratio) on the ordinate measure entry of fluorescent calcium probes into transfected cells. The 3,3-7 (DIPA-1-7) is substantially more potent (˜10× and ˜5×) than 3,4-6 and 3,4-7. Note that 3,4-6 and 3,4-7 species do not reach the same degree maximal efficacy on activation of the receptor, even at supra-maximal concentrations.

FIG. 4. is a graph of fluorescence response (Δ ratio 340/380) in TRPM8 transfected cells as a function of the logarithm of the concentration of the test compound, expressed in μM, for DIPA-1-7 (black circle), 3,4-7 (open squares), or 3,4-6 (open triangles). The assays were conducted by Andersson et al. of King's College, London, UK, using his methods described in “Modulation of the cold-activated channel TRPM8 by lysophospholipids and polyunsaturated fatty acids. Journal Neuroscience 27 (12): 3347-3355, 2007.

In a third set, the selectivity of the test compounds on TRPM8, TRPV1 channels (human TRPV1 gene expressed in HEK293 cells) and TRPA1 channels (human TRPA1 gene expressed in CHO cells) were examined.

The selectivity of DIPA-1-9 on TRP channel receptors, TRPM8, TRPA1 and TRPV1 is shown in Yang et. al. A novel TRPM8 agonist relieves dry eye discomfort. BMC Ophthalmology (21017) 17: 101 (FIG. 3 of manuscript), and incorporated herein by reference. The applicant is a co-author of this publication. This selectivity is also seen with DIPA-1-7 and DIPA-1-8 (data in Wei U.S. Pat. No. 9,956,232, FIG. 1). For these results, the test cells were Chinese Hamster Ovary (CHO) cells or Human Embyronic Kidney (HEK) 293 cells transfected with human TRPV1 or TRPA1 cDNAs. The positive control reference compound was capsaicin (a known TRPV1 agonist) or mustard oil (a known TRPA1 agonist).

In summary, the relative potencies of these test series, as measured by the TRPM8 EC₅₀ [median effective dose], seem to have limited predictive value for comparisons. The 95% Confidence Limits of many EC₅₀ overlap and only analogs with at least a 5-fold difference in potency are clearly distinguishable from each other. To select an ideal ingredient, it is necessary to identify the best cool ingredient and avoid icy cold sensations and adverse tastes. This idea is illustrated in FIG. 5. Furthermore, the duration of action is an important parameter. But the EC₅₀ does not give information on the quality of the heat abstraction sensation, the likelihood of unpleasant taste, or the duration of drug effect. Thus, desirable drug actions (access to and efficacy at TRPM8) are not defined by the EC₅₀. To over-interpret the EC₅₀ is naïve. Other bioassays are required to address the questions of selectivity and specificity. The 3,4-6 and 3,4-7 analogs described as the most active in '496 (Rowsell and Spring, 1978) have weak TRPM8 potencies.

### Study 2

### Irritation Tests

In pre-clinical studies DIPA-1-9 was found not to be irritating when applied to the shaved rat skin at up 20 mg/mL. Injected subcutaneously into the anesthetized rat, DIPA-1-9 did not affect blood pressure or heart rate. DIPA1-9 was applied to the eyelids of patients with dry eye disorder and found not to be irritating. This study has been described in detail in Yang et. al. A novel TRPM8 agonist relieves dry eye discomfort. BMC Ophthalmology (21017) 17: 101, and incorporated herein by reference.

### Study 4

Sensory Qualities of Compounds applied to Oral Cavity and Pharynx

Tests were on six volunteers constituting a “Sensory Panel”, with 3 to 5 trials per substance. Compounds were prepared in water or syrup at 5 mg/mL and administered from a reservoir bottle of 10 mL volume, at a ˜0.3 to 0.5 mL per dose. The delivery was to the base of the tongue (FIG. 3). The subjects asked to rate the sensations for cooling intensity, cold discomfort and adverse taste. Surprisingly, the sensory results were clearcut and there were no ambiguities about the sensory effects that were elicited. The compounds DIPA-1-7, DIPA-1-8, DAPA-2-6, DAPA-2-7 and 3,4-7 produced cold, icy cold, and did not elicit favorable responses. In particular, DIPA-1-7 produced icy pain in the back of the throat and was considered too strong. 3,4-DAPA-6 produced robust cooling, but its duration of action 5 to 10 min, were too short to be of therapeutic value. By contrast, DIPA-1-9 drops produced a coolness and cold which was well-tolerated and the concentration could be increased to 15 mg/mL without objections: that is, there was no pain or discomfort.

The unpleasant tastes produced by DIPA-1-7, DIPA-1-8, 2-6, 2-7, and 2-8 were described as “brackish”, “metallic”, “organic solvent-like”, and “harsh” which lasted for ˜10 to 15 min. The subjects said these taste qualities were undesirable. When tested in the evening near sleep time, the perception of cooling in the throat was more pronounced presumably because there were fewer environmental cues for distraction. In these situations, the heat abstraction sensations were perceived for ≥20 min. Although overt cooling sensation may not be felt after 15 min, the general sense of refreshment in the throat from DIPA-1-9 may persist for 2 to 3+ hours. Surprisingly, increasing the test concentration of DIPA-1-9 from 5 to 8 to 15 mg/mL in drops did not produce icy cold or pain. Thus, there is a safety margin in the use of DIPA-1-9 without risks of a painful throat.

The duration of action DIPA-1-9 was sufficiently long to be of clinical value. The attributes of DIPA-1-9 that makes it selective could not have been predicted from prior art. It was concluded that DIPA-1-9 is the best candidate as an antinociceptive agent for the upper airways.

The 1-di-sec-butyl-phosphorylpentane was also tested in water or syrup, but its duration of action was too short to be of practical value. Like 3,4-DAPA-6 its duration of action was about 5 to 10 min. It is possible that DIPA-1-8 will be a better agent than DIPA-1-9 for situations where there is excessive exudate (mucus and phlegm) in the oropharynx and esophagus, because DIPA-1-8 can more easily reach the TRPM8 receptors in stratum basale than DIPA-1-9. This is also true for the use DIPA-1-7 because its penetration power is better than DIPA-1-9.

### Study 5

### Effects in Rat Model of Swallowing Movements

A principal endogenous irritant in the linings of the upper aerodigestive tract is hydrochloric acid. Acid stimulations of the mucosa of the pharynx will elicit reflex swallowing. Receptive regions are in the pharyngeal walls and innervated by the glossopharyngeal nerve (9^{th}) and the interior superior laryngeal nerve (10^{th}). In a rat animal model, solutions of organic acids such as acetic acid and citric acid were effective in eliciting swallowing [Kajii et al., Sour taste stimulation facilitates reflex swallowing from the pharynx and larynx in the rat Physiology & Behavior 77: 321-325, 2002]. These methods for measuring sensory responses to acid can be adapted for screening the activity of DIPA compounds. An agent that stimulates swallowing or an agent that suppress the acid challenge may then have utility in relieving dysphagia or the discomfort of heartburn, respectively. Preliminary experiments were conducted at the Pavlov Institute of Physiology, St. Petersburg, Russia, using adult male Wistar rats (see Table 11) and Wei (US 2015/0111852 A1). Swallowing movements was identified as the electromyogram activity and could also be visualized as laryngeal movement. Work is in progress to get a complete set of results for DIPA-1-9. It is predicted that DIPA-1-9 may stimulate swallowing at low doses and inhibit acid stimulated swallowing at higher doses.

### Study 6

### Pre-Clinical Studies of Mouse Cough Model of Upper Respiratory Tract Infection

This study was conducted at the State Key Laboratory of Respiratory Disease,

Guangzhou Institute of Respiratory Disease, Guangzhou Medical University, Guangzhou, China The investigators were Ren Nee, Dong PeiJian, Liu ChunLi, Zhang Qingling, Wei TakFung, and Zhong NanShan. The methods used described earlier. Ye X M et al. Zhonghua Yi Xue Za Zhi. (2011) 91(24):1708-12. [A guinea pig model of respiratory syncytial virus infection for cough and its neurogenic inflammatory mechanism] Chinese. Ye X M et al. Cough reflex sensitivity is increased in guinea pigs with parainfluenza virus infection. Exp Lung Res. (2011) 37(3):186-94. Mice were used instead of guinea pigs. The experimental procedures were approved by the Institutional Animal Care and Use Committee.

Briefly, mice were intranasally inoculated with respiratory syncytial virus (RSV) and the cough count monitored with an Buxco system (Buxco, Wilmington, N.C., USA). The dosing parameters were as: 25 μL intranasal instillation per mouse for saline and DIPA-1-9 (20 mg/mL), 0.1 mL per mouse for perioral codeine, 10 mg/mL. Cough counts were measured 10 min after saline or DIPA-1-9 and 1 hr after codeine. Cough frequency was detected as a transient change in airflow pressure in a chamber and the signal recorded via a pressure transducer and computer. Additionally, the audio-amplified count was also recorded electronically. Coughs were counted for the 6 min. The experiment was visually monitored by the investigator. As shown earlier, peak cough frequency approximately 2 weeks after inoculation, when viral replication and airway pathology is verified by RSV RNA measurements, cytology and histopathology. The course of airway inflammation diminishes by 4 to 7 weeks after inoculation and mimics human respiratory tract infections.

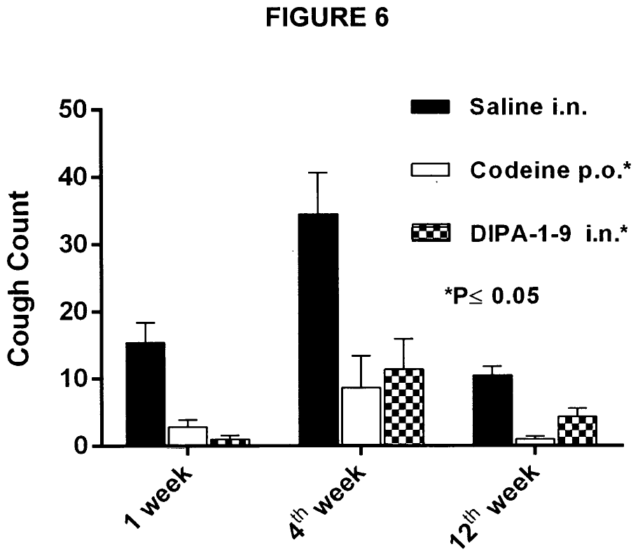

FIG. 6. shows DIPA-1-9 inhibits cough frequency in a mouse model of respiratory tract viral infection. Mice (n=4 to 6 per group) cough more frequently (black bars) after inoculation with respiratory synctial virus (RSV). Codeine administered 1 mg perioral (p.o.) per mouse, or DIPA-1-9 0.5 mg in 25 μL intranasally (i.n.) per mouse, significantly inhibited cough frequency (*P≤0.01 and ≤0.05 for the three time periods of testing, Dunnett's test for multiple comparison). These results in mice show that DIPA-1-9 has potential antinociceptive activity in the upper aerodigestive tract.

### Case Studies

The rationale and data set for selecting DIPA-1-9 in drops as a treatment agent for airway disorders have been described. In the case studies reported herein, the efficacy of DIPA-1-9 was investigated in volunteers for: a) control of acute cough, b) control of chronic cough, c) control of cough hypersensitivity, d) facilitation of mucus expectoration in a case of productive cough, h) reduction of the sense of dyspnea and for insomnia.

The drops were easy to use. An effect consistently observed was a rapid onset (≤2 min) of the sensation of coolness in the throat after application of DIPA-1-9 drops to the base of the tongue. The coolness spreads to the rest of throat and intensifies, as if a spoonful of rich ice cream had been swallowed. This cooling effect lasts for ≥15 min, and any discomfort in the throat is relieved. The cooling sensation can be used to facilitate mucus expectoration from the airways. Also relieved is the sense of suffocation when lying down to sleep in a subject that has dyspnea. Sleep is facilitated. The drops do not have adverse tastes when formulated with an artificial sweetener, or produce cold discomfort behind the sternum.

### Case 1.

Two cases of subjects with cough variant asthma (CVA) are described here. CVA is a type of asthma in which the main symptom is a persistent non-productive cough, i.e. a cough that does not produce mucus. The cough, by definition of the condition, persists for at least 8 weeks and may be aggravated by such conditions as dry, smoky air, or respiratory tract infections. Treatment with normal asthma medications such as inhaled steroids and beta-adrenergic agonists (to relax bronchial smooth muscle) have limited value in reducing the cough of cough variant asthma.

The first subject was a 25-year old male working in a diner serving kebabs and grilled meat in the South of France. Business was good but he worked in a smoky environment and developed a persistent cough that lasted for 6+ months. He was diagnosed as having CVA, but standard medications for asthma did not affect the frequency of coughing which was constant, debilitating, and affected his ability at work. He was distressed because his physician's advice and prescriptions were not working. The subject agreed to try the cough syrup and was given a packet of 20 vials, each vial containing 1 mL of DIPA-1-9, 8 mg/mL dissolved in cherry-flavored syrup. He was instructed to use the vials on an as-needed basis to reduce the urge to cough, but not to exceed 3 vials per day. Surprisingly, the subject noted that the cough frequency went down within 3 days of use and was not bothersome after one week. He asked for a continued supply of the vials which was given to him, but after one month the subject declared that the coughing problem had disappeared. He was most grateful for the opportunity to try the DIPA-1-9.

A 72-year old male, prominent in business circles in Hong Kong, developed a persistent cough. He was a smoker and had allergic rhinitis, but did not manifest wheezing upon exertion. He was misdiagnosed as having tuberculosis, and put on a course of isoniazid and other drugs. He lost weight and became apprehensive about his future. His cough occurred spontaneously and did not need triggers, but the cough frequency increased with socializing, with drinking, laughing and speaking. This cough was present for 3+ months and did not to go away. His doctor changed his diagnosis to asthma and prescribed Singulair, but this did not work. After a particular embarrassing episode, when he coughed violently after eating a piece of Szechuan pepper fish during a banquet, the subject volunteered to try an experimental remedy. He was given two packets of DIPA-1-9 vials, each vial containing ten 1 mL of DIPA-1-9, 8 mg/mL in simple syrup. He consumed the vials within 5 days and asked for more. This regimen was repeated for another 5 days, and surprisingly the cough was gone. He said that he had always been skeptical of academic scientists because such people did not seem to him to do anything significant, but this time he was happy to participate in an experiment.

In these two cases of chronic cough, DIPA-1-9 in syrup was effective for cough suppression but also appeared to act by reducing the cough hypersensitivity syndrome: i.e. over time the nerve endings became less sensitive to tussive stimuli. The subjects became more optimistic as the coughing urge and frequency was brought under control. They became less paranoid about progression of a serious illness. Their ability to socialize increased. The ability of the DIPA-1-9 syrup to relieve throat discomfort was self-evident and robust. The case with the cough variant asthma was interesting because the disorder was in the lower airways, yet the administration of a TRPM8 agonist to the upper airways controlled the symptoms, and the subject was completely recovered.

### Case Study 2

A 50-year old male scientist received an award to conduct a 6-month research project in Guangzhou, China. He rented a hotel room and lived alone. He used the public subway and, in the fall, he “caught the flu” with a 3-day fever and throat discomfort, chills and coughing. He developed a “productive” cough with thick mucus, which gradually thinned out after about a week, but the cough persisted and increased in frequency, until his throat felt raw. The presence of mucus also continued, although it did not become purulent. He did a count on his coughing and reported an averaged of 25 to 40 coughs per hr, with higher frequency at night. He could not sleep well because lying down on the bed exacerbated the itch in his throat and increased the urge to cough. Because he worked in the laboratory, he had access to the DIPA-1-9 syrup (Simple Syrup, 8 mg/mL stored 0.8 mL per plastic vial) and began to experiment on himself. He took the syrup on an as needed basis for three successive days and used two to three vials per day. He said that the cough frequency went down to an average of 5 to 10 coughs per hour. He said he slept better than he had in the two preceding weeks. He remarked that he learned how to utilize the DIPA-1-9 syrup to help expectorate mucus in his airways. He said that: “Instead of letting the itch in my throat stimulate non-productive coughs, I will make use of the cooling effect of the syrup to suppress the urge the cough until I could feel a lump of mucus gradually accumulate in my throat. Then I will go to the bathroom, stand over the sink, brace myself with my arms on the rim of the sink, and heave out the phlegm. The coolness in my throat allowed me to this without significant discomfort to my throat lining. A second method of heaving was to stand over the toilet, place my hands on top of my upper legs and heave into the toilet. Getting rid of the mucus felt good! It was particularly important in helping me having a good night's sleep.” After using the syrup for 5 days, the cough and throat discomfort disappeared.

This experience was repeated with two other subjects that had influenza and developed productive cough. In these two subjects, the drops comprised of DIPA-1-9 dissolved 5 mg/mL in water, plus the artificial sweeter sucralose at 2 mg/mL or acesulfame-K at 5 mg/mL. In one subject, pharyngitis and productive cough was of 2+ weeks duration. These cases illustrate the value of the DIPA-1-9 drops in helping the subject expectorate phlegm. Mucus clearance is an important therapeutic goal in the treatment of airway inflammation. If the airway inflammation cannot be ameliorated then the mucus accumulation exacerbates the airway injury and may even threaten the patient's life. The progressive movement of mucus up the airways triggers the cough. But frequently the cough removal of mucus is “not efficient”, i.e. it does not remove the mucus, and the throat lining becomes raw and painful from the coughing effort. The cooling actions of the DIPA-1-9 enable the subject to suppress the urge to cough until there is sufficient mucus to expectorate. Thus, the efficiency of mucus clearance is increased.

### Case Study 3

A retired clinical pharmacologist worked at an out-patient clinic and consulted patients with respiratory problems. He frequently saw patients with cough, and he was atuned to current research, but he felt that the pipeline drugs were probably too costly for the treatment of acute cough. He volunteered to test the DIPA-1-9 formulations after obtaining informed consent from his subjects. Over a 3-month period, he recruited and made observations on 10 subjects with cough using a standardized questionnaire. There were 3 M, 7 F in the group, average age of 46 years, with cough of: unknown etiology (4), post-infectious cough (4), one bronchitis, and one eosinophilic bronchitis. Subjects were given a sprayer containing DIPA-1-9, 5 mg/mL in syrup, and a questionnaire to self-report cough frequency over a period of 1 week. At the end of the test period, the subjects reported that the medication was: very effective (3), partially effective (4), and not effective (3). All 3 of the “not effective” subjects came from the group with cough of “unknown etiology”.

### Case Study 4

A distinguished Professor of Pharmacology and Respiratory Medicine became interested in the use of cooling agents for cough and for clearance of airway mucus. In his group of 10 graduate students and post-doctoral fellows, 5 had episodes of coughing and found the DIPA-1-9 syrup combination to be clearly efficacious in the treatment of their coughing discomfort. One graduate student even tested it on her grandmother and found that it worked. Asked to comment on the mechanisms of action of DIPA-1-9, the Professor noted: “The primary goal is always to have the right molecule delivered to the right place at the right dose. Here, placement of DIPA-1-9 on the receptive field of the 9^{th} nerve is important. Direct delivery to the 10^{th} nerve afferents will most likely evoke coughing. It is well-known that cold air will evoke coughing in asthma patients, and this may be a 10^{th} nerve phenomenon. Using the syrup and avoiding aerosol droplet contact to the laryngeal afferents is an imaginative step. If the DIPA-1-9 syrup works, it will be a significant advance, but don't expect too much credit. People will say it is obvious because menthol lozenges are used for cough. On the other hand, I think menthol lozenges work because they are sweet, and the sweetened saliva has to be constantly swallowed. In a menthol lozenge it is the swallowing of the sweetened saliva that stops the cough, not necessarily the cooling actions of menthol which are limited.”

### Case Study 5

A 75-year old retired engineer had Parkinson's disease for 20 years. He had the best medical care which included brain stimulation of the thalamus but in the past two years his motor abilities deteriorated and he complained of poor sleep, muscle rigidity, and difficulty in chewing and swallowing food, but his most distressing symptoms were labored breathing and panic attacks arising from thoughts of suffocation. He volunteered to use the DIPA-1-9 syrup, 8 mg/mL stored 0.8 mL in a plastic vial, before going to sleep. His wife immediately noticed that he fell asleep quickly and slept without interruption until morning. The subject continued to use the DIPA-1-9 on an as-needed basis. He said the syrup gave him a refreshing sensation in the throat and a sense of relaxed breathing of cool air without effort. He could chew and swallow his food comfortably. The fear of suffocation at night disappeared. His panic attacks have also not reappeared.

A 72-year female subject had chronic obstructive pulmonary disease for over 15 years. The primary cause of her condition was initially asthma, with seasonal bouts, but after a particular episode when she developed bronchitis, the condition worsened to COPD. She volunteered to try the DIPA-1-9, formulated 5 mg/mL in water, on an as needed basis. She said the drops helped her sleep better and breathe more freely at night, especially when she woke up with coughing and choking sensations. She has continued the DIPA-1-9 drops for over a period of six months without incident.

In summary, the concept has been put forward that heat abstraction sensations, captured by topical application of a molecule designed to selectively activate TRPM8, can be used to alleviate discomforts of the airway disorders. By synthesizing compounds and devising tests, a molecule named DIPA-1-9 was identified as having the properties for achieving the desired sensory effect: namely, an ideal cool sensation equivalent to that of a spoonful of a rich ice cream, on the oropharynx. On receptor targets, DIPA-1-9 was selective for TRPM8 and not TRPVI and TRPA1.

When bioassayed in mice, these compounds inhibited acid-induced swallowing and virus-induced cough. These studies in animals showed that DIPA-1-9 has an anti-irritant, or an antinociceptive action. The water solubility of DIPA-1-9 facilitates its homogeneous dissolution in water or syrup for localized delivery to the pharyngeal surface. The volume of the drops times the concentration of DIPA-1-9 is equal to the dose. A dose of DIPA-1-9 of about 1 to 5 mg in a volume of <0.05 mL of vehicle delivered to the base of the tongue will produce a robust, cooling sensation, without irritation and sting, and without unpleasant taste, lasting ≥15 min. The onset of drug action of ≤2 min. This immediate onset is surprising and unprecedented as there are no similar products on the market. Patients want their symptoms of throat discomfort relieved quickly after self-administration of an agent. The rapid onset allows the subject to control the urge to cough. The DIPA-1-9 sensory effect is sufficient to treat discomforts of the lower airways, including: acute and chronic cough from airway irritation or inflammation. The drug mechanism of controlling the urge to cough allows the subject to increase mucus clearance from the airways, which is an important therapeutic goal. The DIPA-1-9 drops applied to the upper airways can also be used to alleviate dyspnea. In summary, DIPA-1-9 formulated in water or syrup and delivered in a volume of 50.5 mL per unit dose to the base of the tongue is an ideal rapid onset medication for reducing sensory discomfort of the lower airways in a subject in need of treatment, and use of this formulation may have value for therapeutic treatment of lower airway blockage diseases.

1. A therapeutic method for the treatment of a lower airways disorder in a subject in need of such treatment, comprising:
providing a composition including 1-[Diisopropyl-phosphinoyl]-nonane dissolved in a pharmaceutical vehicle, the 1-[Diisopropyl-phosphinoyl]-nonane being in a therapeutically effective amount for treating a lower airways disorder when topically applied to an upper airways site; and
topically applying said composition to the oropharynx as the upper airways site.

2. The method as in claim 1 wherein the lower airway disorder comprises cough, inflammation of the airways, chronic obstructive pulmonary disease, mucus accumulation in the airways, cystic fibrosis, idiopathic pulmonary fibrosis, interstitial lung diseases, bronchitis, bronchiectasis, and the condition known as asthma.

3. The method as in claim 1 wherein the lower airway disorder comprises chronic obstructive pulmonary disease, chronic obstructive airway disease, or chronic obstructive lung disease.

4. The method as in claim 1 wherein the lower airway disorder is dyspnea and a sense of suffocation.

5. The method as in claim 1 wherein the lower airway disorder is a shortness of breath.

6. The method as in claim 1 wherein the lower airway disorder is sleep apnea.

7. The method as in claim 1 wherein the composition applied is to the base of the subject's oropharynx and is in a volume of about 0.05 to 0.5 mL per unit dose.

8. The method as in claim 7 wherein the 1-[Diisopropyl-phosphinoyl]-nonane is dissolved in the vehicle at a concentration therein of 1 to 15 mg/ml and the vehicle comprises water, a water-based solution, or a syrup.

9. The method as in claim 1 wherein the pharmaceutical vehicle comprises water or a water-based solution and an artificial sweetener (sucralose or acesulfame-K).
